(19) United States
(12) Patent Application Publication
(10) Pub. No.: US 2023/0018980 A1
(43) Pub. Date: Jan. 19, 2023

(54) BISFLUOROALKYL-1,4-BENZODIAZEPINONE COMPOUNDS FOR TREATING DESMOID TUMORS

(71) Applicants: Bristol-Myers Squibb Company, Princeton, NJ (US); Ayala Pharmaceuticals Inc., Wilmington, DE (US)

(72) Inventors: Bruce S. FISCHER, East Brunswick, NJ (US); Gaurav Bajaj, Plainsboro, NJ (US); Matti Davis, Modiin (IL); Joel Kaye, Netanya (IL)

(73) Assignees: Bristol-Myers Squibb Company, Princeton, NJ (US); Ayala Pharmaceuticals Inc., Wilmington, DE (US)

(21) Appl. No.: 17/942,217

(22) Filed: Sep. 12, 2022

Related U.S. Application Data
(63) Continuation-in-part of application No. 17/857,277, filed on Jul. 5, 2022.
Application No. 17/857,277 is a continuation-in-part of application No. 17/077,857, filed on Oct. 22, 2020.
Application No. 17/077,857 is a continuation-in-part of application No. PCT/US2019/030771, filed on May 5, 2019.
Application No. PCT/US2019/030771 is a continuation-in-part of application No. 17/052,812, filed on Nov. 4, 2020.
Application No. 17/052,812 is a continuation-in-part of application No. PCT/US2022/028956, filed on May 12, 2022.
(60) Provisional application No. 62/667,540, filed on May 6, 2018.
(60) Provisional application No. 63/187,991, filed on May 13, 2021.
(60) Provisional application No. 63/214,355, filed on Jun. 24, 2021.

(51) Int. Cl. A61K 31/5513; A61K 9/00; A61P 35/00

(52) U.S. Cl. CPC A61K 31/5513; A61K 9/0056; A61P 35/00

(57) ABSTRACT

The present invention provides compositions comprising bisfluoroalkyl-1,4-benzodiazepinone compounds, including compounds of Formula (I) or prodrugs thereof;

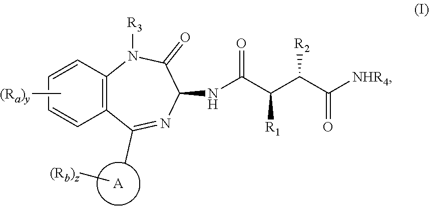

optionally in combination with an additional cancer therapeutic agent, and methods of use thereof for treating proliferative diseases and disorders such as Desmoid tumors.

### CROSS REFERENCE TO RELATED APPLICATIONS

This patent application is a continuation-in-part of U.S. patent application Ser. No. 17/857,277 filed on Jul. 5, 2022, which is a continuation-in-part of U.S. patent application Ser. No. 17/077,857 filed on 22 Oct. 2020, which is a continuation-in-part of International Patent Application Serial No. PCT/US2019/030771 filed on 5 May 2019, which claims the benefit of U.S. Provisional Patent Application Ser. No. 62/667,540 filed on 6 May 2018. This patent application is also a continuation-in-part of U.S. patent application Ser. No. 17/052,812 filed on 4 Nov. 2020, which is a National Phase application of International Patent Application Serial No. PCT/US2019/030771 filed on 5 May 2019, which claims the benefit of U.S. Provisional Patent Application Ser. No. 62/667,540 filed on 6 May 2018. This patent application is also a continuation-in-part of International Patent Application Serial No. PCT/US2022/028956 filed on 12 May 2022, which claims the benefit of U.S. Provisional Patent Application Ser. No. 63/187,991 filed on 13 May 2021 and U.S. Provisional Patent Application Ser. No. 63/214,355 filed on 24 Jun. 2021. All of the applications listed hereinabove are hereby incorporated by reference herein in their entirety.

### FIELD OF THE INVENTION

The present invention provides compositions comprising bisfluoroalkyl-1,4-benzodiazepinone compounds, including compounds of Formula (I) or prodrugs thereof;

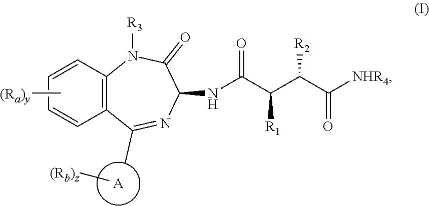

optionally in combination with an additional cancer therapeutic agent, and methods of use thereof for treating proliferative diseases and disorders such as Desmoid tumors.

### BACKGROUND OF THE INVENTION

Desmoid tumors, also called aggressive fibromatosis or desmoid-type fibromatosis, are rare connective tissue tumors that typically arise in the upper and lower extremities, abdominal wall, head and neck area, mesenteric root and chest wall with the potential to arise in additional parts of the body. According to the World Health Organization, desmoid tumors are defined as “clonal fibroblastic proliferations that arise in the deep soft tissues and are characterized by infiltrative growth and a tendency toward local recurrence but an inability to metastasize.”

Desmoid tumors do not metastasize, but often aggressively infiltrate neurovascular structures and vital organs. People living with desmoid tumors are often limited in their daily life due to chronic pain, functional deficits, general decrease in their quality of life and organ dysfunction. Desmoid tumors have an annual incidence of approximately 1,700 patients in the United States and typically occur in patients between the ages of 15 and 60 years. They are most commonly diagnosed in young adults between 30-40 years of age and are more prevalent in females. There is a high rate of recurrence post-surgery, and there are currently no FDA-approved systemic therapies for the treatment of unresectable, recurrent or progressive desmoid tumors.

### SUMMARY OF THE INVENTION

In one embodiment, the present invention provides a method of treating a Desmoid tumor or inhibiting the growth of a Desmoid tumor in a subject comprising the step of administering to said subject a composition comprising one or more compounds represented by the structure of Formula (I):

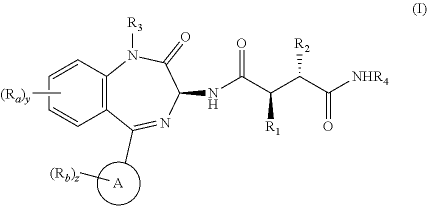

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

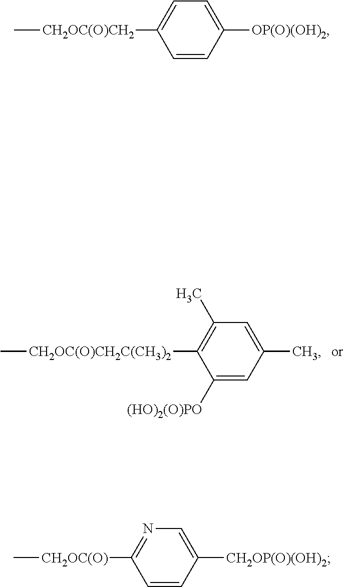

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2.

In some embodiments, the composition is administered at a dose of 1.2 mg, 2 mg, or 4 mg.

In some embodiments, the composition is administered at a dose of 1.2 mg daily.

In some embodiments, the composition is administered intermittently at a dose of 2 mg or 4 mg. In some embodiments, the intermittent administration comprises 2 days of administration followed by 5 days where the subject is not administered the treatment.

In some embodiments, the compound of Formula (I) comprises:

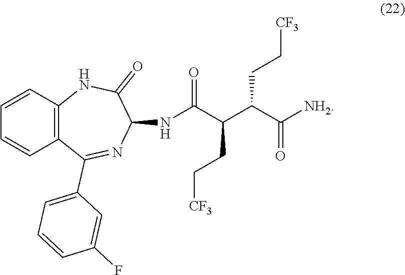 [SMILES: NC(=O)[C@@H](CCC(F)(F)F)[C@@H](CCC(F)(F)F)C(=O)N[C@H]1N=C(c2cccc(F)c2)c2ccccc2NC1=O]

In some embodiments, the composition is orally administered to the subject.

### DETAILED DESCRIPTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

### Methods of Use

In one embodiment, the present invention provides a method of treating, suppressing or inhibiting a hyperproliferative disorder in a subject comprising the step of administering to said subject a composition comprising one or more compounds represented by the structure of Formula (I):

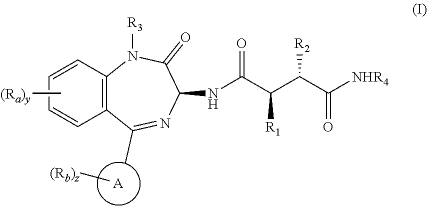

and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

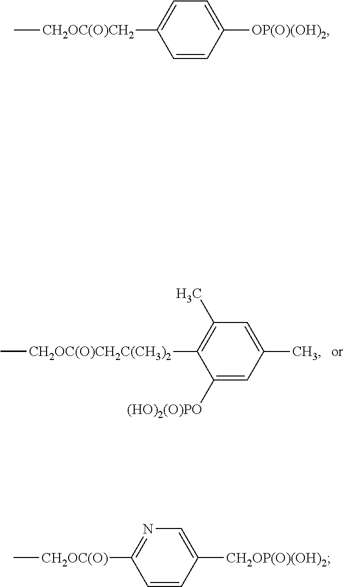

R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2.

In one embodiment, the present invention provides the use of the compounds, compositions, or combinations as described herein for treating, suppressing or inhibiting a proliferative disorder in a subject. In some embodiments, the proliferative disorder is a Desmoid tumor. In some embodiments, the proliferative disorder is aggressive fibromatosis. In some embodiments, the proliferative disorder is desmoid-type fibromatosis.

In some embodiments, the Desmoid tumor is a sporadic desmoid tumor manifestation. In other embodiments, the Desmoid tumor is present in a subject having familial adenomatous polyposis (FAP). In other embodiments, the Desmoid tumor is present in a subject with Gardner syndrome.

In one embodiment, the present invention provides a method of treating a Desmoid tumor disorder in a subject, comprising the step of administering to the subject a composition comprising one or more compounds of Formula (I) or prodrugs or salts thereof, as described herein. In another embodiment, the present invention provides a method of suppressing or inhibiting a Desmoid tumor in a subject, comprising the step of administering to the subject a composition comprising one or more compounds of Formula (I) or prodrugs or salts thereof, as described herein.

In one embodiment, the present invention provides a method of treating a Desmoid tumor disorder in a subject, comprising the step of administering to the subject a composition comprising one or more compounds of Formula (II) or prodrugs or salts thereof, as described herein. In another embodiment, the present invention provides a method of suppressing or inhibiting a Desmoid tumor in a subject, comprising the step of administering to the subject a composition comprising one or more compounds of Formula (II) or prodrugs or salts thereof, as described herein.

In one embodiment, the present invention provides a method of treating a Desmoid tumor disorder in a subject, comprising the step of administering to the subject a composition comprising one or more compounds of Formula (III) or prodrugs or salts thereof, as described herein. In another embodiment, the present invention provides a method of suppressing or inhibiting a Desmoid tumor in a subject, comprising the step of administering to the subject a composition comprising one or more compounds of Formula (III) or prodrugs or salts thereof, as described herein.

In one embodiment, the present invention provides a method of treating a Desmoid tumor disorder in a subject, comprising the step of administering to the subject a composition comprising one or more compounds of Formula (IV) or prodrugs or salts thereof, as described herein. In another embodiment, the present invention provides a method of suppressing or inhibiting a Desmoid tumor in a subject, comprising the step of administering to the subject a composition comprising one or more compounds of Formula (IV) or prodrugs or salts thereof, as described herein.

In one embodiment, the present invention provides a method of treating a Desmoid tumor disorder in a subject, comprising the step of administering to the subject a composition comprising one or more compounds of Formula (V) or prodrugs or salts thereof, as described herein. In another embodiment, the present invention provides a method of suppressing or inhibiting a Desmoid tumor in a subject, comprising the step of administering to the subject a composition comprising one or more compounds of Formula (V) or prodrugs or salts thereof, as described herein.

In another embodiment, the present invention provides a method of suppressing or inhibiting tumor growth in a subject, comprising the step of administering to the subject a composition comprising one or more compounds of Formula (I) or Formula (IV) or prodrugs or salts thereof, as described herein. In another embodiment, tumor growth is suppressed by 20-35%. In another embodiment, tumor growth is suppressed by 35-50%. In another embodiment, tumor growth is suppressed by 50-75%. In another embodiment, tumor growth is suppressed by 75-90%. In another embodiment, tumor growth is suppressed by 90-99%.

In another embodiment, tumor growth is suppressed by 20%. In another embodiment, tumor growth is suppressed by 25%. In another embodiment, tumor growth is suppressed by 30%. In another embodiment, tumor growth is suppressed by 35%. In another embodiment, tumor growth is suppressed by 40%. In another embodiment, tumor growth is suppressed by 45%. In another embodiment, tumor growth is suppressed by 50%. In another embodiment, tumor growth is suppressed by 55%. In another embodiment, tumor growth is suppressed by 60%. In another embodiment, tumor growth is suppressed by 65%. In another embodiment, tumor growth is suppressed by 70%. In another embodiment, tumor growth is suppressed by 75%. In another embodiment, tumor growth is suppressed by 80%. In another embodiment, tumor growth is suppressed by 85%. In another embodiment, tumor growth is suppressed by 90%. In another embodiment, tumor growth is suppressed by 95%. In another embodiment, tumor growth is suppressed by 99%.

In another embodiment, the present invention provides a method of decreasing tumor size or tumor volume in a subject having a hyperproliferative disorder, comprising the step of administering to the subject a composition comprising one or more compounds of Formula (I) or Formula (IV) or prodrugs or salts thereof, as described herein.

In one embodiment, reducing tumor size or volume comprises decreasing tumor size or volume by 25%-95%. In another embodiment, reducing tumor size or volume comprises decreasing tumor size or volume by 25%. In another embodiment, reducing tumor size or volume comprises decreasing tumor size or volume by 30%. In another embodiment, reducing tumor size or volume comprises decreasing tumor size or volume by 35%. In another embodiment, reducing tumor size or volume comprises decreasing tumor size or volume by 40%. In another embodiment, reducing tumor size or volume comprises decreasing tumor size or volume by 45%. In another embodiment, reducing tumor size or volume comprises decreasing tumor size or volume by 50%. In another embodiment, reducing tumor size or volume comprises decreasing tumor size or volume by 55%. In another embodiment, reducing tumor size or volume comprises decreasing tumor size or volume by 60%. In another embodiment, reducing tumor size or volume comprises decreasing tumor size or volume by 65%. In another embodiment, reducing tumor size or volume comprises decreasing tumor size or volume by 70%. In another embodiment, reducing tumor size or volume comprises decreasing tumor size or volume by 75%. In another embodiment, reducing tumor size or volume comprises decreasing tumor size or volume by 80%. In another embodiment, reducing tumor size or volume comprises decreasing tumor size or volume by 85%. In another embodiment, reducing tumor size or volume comprises decreasing tumor size or volume by 90%. In another embodiment, reducing tumor size or volume comprises decreasing tumor size or volume by 95%.

In another embodiment, the present invention provides a method of inhibiting the progression of a hyperproliferative disorder, a method of decreasing tumor diameter, a method of decreasing tumor volume, a method of inhibiting an increase in tumor diameter or tumor volume. In one embodiment, the inhibition of tumor growth using the methods as described herein may be seen during the period in which the compounds of the present invention are administered. In another embodiment, the inhibition may be seen after the last dose of the treatment as described herein.

In another embodiment, the present invention provides a method of avoiding resistance to therapy in a subject having a hyperproliferative disorder, comprising the steps of administering to the subject a first composition comprising one or more compounds of Formula (I) or Formula (IV) or prodrugs or salts thereof, as described herein.

In some embodiments, resistance to therapy comprises resistance to radiation therapy, chemotherapy, immunotherapy, hormone therapy, radiation, or photodynamic therapy.

In another embodiment, the present invention provides a method of delaying, inhibiting, or suppressing relapse of an active hyperproliferative disorder in a subject, comprising the step of administering to the subject a composition comprising one or more compounds of Formula (I) or Formula (IV) or prodrugs or salts thereof, as described herein.

In another embodiment, the present invention provides a method of delaying, inhibiting, or suppressing relapse to a hyperproliferative disorder in a subject, comprising the step of administering to the subject a composition comprising one or more compounds of Formula (I) or Formula (IV) or prodrugs or salts thereof, as described herein.

In another embodiment, the present invention provides a method of treating a hyperproliferative disorder with minimal side effects in a subject, comprising the step of administering to the subject a composition comprising one or more compounds of Formula (I) or Formula (IV) or prodrugs or salts thereof, as described herein.

In some embodiments, a side effect comprises a weight change. In some embodiments, a side effect comprises weight loss. In some embodiments, a side effect comprises weight gain. In some embodiments, weight change is associated with loss of appetite, nausea, diarrhea, or vomiting.

In another embodiment, the present invention provides a method of prolonging relapse-free survival, progression-free survival, overall survival, or a combination thereof in a subject having a hyperproliferative disorder, comprising the steps of administering to the subject a composition comprising one or more compounds of Formula (I) or Formula (IV) or prodrugs or salts thereof, as described herein.

In another embodiment, the present invention provides a method of increasing or lengthening survival of a subject having a hyperproliferative disorder, comprising the steps of administering to the subject a first composition comprising one or more compounds of Formula (I) or Formula (IV) or prodrugs or salts thereof, as described herein.

In another embodiment, the present invention provides a method of inhibiting cell migration in a subject having a hyperproliferative disorder comprising the steps of administering to the subject a first composition comprising one or more compounds of Formula (I) or Formula (IV) or prodrugs or salts thereof, as described herein.

In another embodiment, the present invention provides a method of inhibiting cell invasion in a subject having a hyperproliferative disorder comprising the steps of administering to the subject a first composition comprising one or more compounds of Formula (I) or Formula (IV) or prodrugs or salts thereof, as described herein.

In another embodiment, the present invention provides a method of delaying, inhibiting, or suppressing metastasis of a hyperproliferative disorder in a subject, comprising the step of administering to the subject a composition comprising one or more compounds of Formula (I) or Formula (IV) or prodrugs or salts thereof, as described herein.

In another embodiment, the present invention provides a method of downregulating the expression of Notch-related genes in a subject having a hyperproliferative disorder, comprising the steps of administering to the subject a first composition comprising one or more compounds of Formula (I) or Formula (IV) or prodrugs or salts thereof, as described herein.

In one embodiment, the Notch-related gene that is downregulated comprises HIF1A, HES2, HES5, HEYL, HEY1, HEY2, NRARP, BCL2A1, MYC, or a combination thereof.

In another embodiment, the present invention provides a method of upregulating the expression of Notch-related genes in a subject having a hyperproliferative disorder, comprising the steps of administering to the subject a first composition comprising one or more compounds of Formula (I) or Formula (IV) or prodrugs or salts thereof, as described herein.

In some embodiments, the Notch-related genes comprise Notch target genes. In some embodiments, the Notch-related genes comprise genes downstream of Notch in the Notch signaling pathway. In some embodiments, the Notch-related genes comprise Notch-regulated genes.

In some embodiments, the Notch-related genes comprise HES2, HES4, HES5, HEYL, HEY1, HEY2, NRARP, BCL2A1, HIF1A, or a combination thereof. In some embodiments, the Notch-related genes comprise HES1, MYC, or a combination thereof.

In another embodiment, the methods as provided herein comprise the step of administering to the subject a composition comprising one or more compounds of Formula (III) or prodrugs or salts thereof, as described herein. In one embodiment, the compound of Formula (I) comprises a compound of Formula (III) as described herein. In one embodiment, the compound of Formula (III) comprises Compound (1) as described herein. In another embodiment, the compound of Formula (III) comprises Compound (2) as described herein.

In other embodiments, the compound of Formula (I) comprises a compound of Formula (IV) as described herein. In one embodiment, the compound of Formula (IV) comprises Compound (22) as described herein.

In some embodiments, “downregulating” or “upregulating” the expression of a Notch-related gene may encompass the gene's RNA level relative to its RNA level at a different time, in a different tissue or cell (which, in some embodiments, is a non-tumor tissue or cell), or in other subjects, and is presented as fold change. For example, in some embodiments, determining that the expression of a gene is downregulated or upregulated is accomplished by comparing the RNA level of the gene prior to treatment with the RNA level of the gene after treatment. A skilled artisan would be knowledgeable of the tools and methods available for determining gene expression levels, or RNA levels, for example, by RT-PCR.

In some embodiments, the present invention provides a method of inhibiting, treating or suppressing a hyperproliferative disorder in a subject, comprising the steps of: (a) administering to the subject a composition comprising one or more compounds of Formula (I) or Formula (IV) or prodrugs or salts thereof, as described herein; (b) measuring plasma total free active concentration of said compound of Formula (I) or Formula (IV); and (c) adjusting the administered dose of said compound of Formula (I) or Formula (IV).

In some embodiments, adjusting the administered dose comprises:
i. if the plasma total free active concentration of said compound of Formula (I) or Formula (IV) of said subject is at the desired concentration then the dose administered is continued;
ii. if the plasma total free active concentration of said compound of Formula (I) or Formula (IV) of said subject is below the desired concentration then the dose administered is increased; and
iii. if the plasma total free active concentration of said compound of Formula (I) or Formula (IV) of said subject is above the desired concentration then the dose administered is decreased.

A skilled artisan would be knowledgeable of the tools and methods available for determining measuring plasma total free active concentration of said compound of Formula (I) or Formula (IV). In some embodiments, the “desired concentration” is the effective concentration (EC), which may encompass a dose or concentration of a drug that produces a biological response, which in some embodiments, is effective in reducing tumor size or inhibits tumor growth.

It is to be understood that each of these methods is a separate embodiment of the invention.

In another embodiment, the present invention provides a method of treating, suppressing or inhibiting a hyperproliferative disorder in a subject comprising the step of administering to the subject a composition consisting essentially of one or more compounds represented by the structure of Formula (I) or Formula (IV) as described hereinabove, and, optionally, a second composition as described herein. In another embodiment, the present invention provides a method of treating, suppressing or inhibiting a hyperproliferative disorder in a subject comprising the step of administering to the subject a composition consisting of one or more compounds represented by the structure of Formula (I) or Formula (IV) as described hereinabove, and, optionally, a second composition as described herein.

In one embodiment, the present invention provides the use of a therapeutically acceptable amount of the compound(s) as described herein for treating, suppressing or inhibiting a hyperproliferative disorder in a subject. In another embodiment, the present invention provides the use of a therapeutically effective amount of the compound(s) as described herein for treating, suppressing or inhibiting a hyperproliferative disorder in a subject. In another embodiment, the present invention provides the use of a synergistically effective amount of the compound(s) as described herein for treating, suppressing or inhibiting a hyperproliferative disorder in a subject. In another embodiment, the present invention provides the use of a synergistically therapeutically effective amount of one or more compound(s) as described herein for treating, suppressing or inhibiting a hyperproliferative disorder in a subject.

In other embodiments, the present invention provides compounds, compositions or compounds as described herein for use in any of the methods as described herein, including, inter alia, treating, suppressing or inhibiting a hyperproliferative disorder in a subject. In other embodiments, the present invention provides compounds, compositions or compounds as described herein for the preparation of a medicament for any of the methods as described herein, including, inter alia, treating, suppressing or inhibiting a hyperproliferative disorder in a subject.

In some embodiments, the methods of the present invention further comprise the step of identifying a candidate subject for treatment with a first composition comprising one or more compounds represented by the structure of Formula (I) or Formula (IV), as described herein, comprising the step of evaluating Notch gene function in the subject. In one embodiment, evaluating Notch gene function comprises determining if there are Notch mutations. In one embodiment, the Notch mutations are in a PEST region of a Notch gene. In another embodiment, the Notch mutations are in the NRR of a Notch gene. In another embodiment, evaluating Notch gene function comprises determining the expression of Notch-regulated genes. In one embodiment, the genes are downstream of Notch in the Notch signaling pathway.

In some embodiments, evaluating Notch gene function comprises RNA-seq or another RNA sequencing tool to reveal the presence and quantity of RNA in a biological sample at a given moment. In another embodiment, other methods of evaluating the quantity of downstream Notch protein RNA may be utilized, as are well known in the art. In another embodiment, the evaluator comprises a DNA sequencing method, as are known in the art.

In one embodiment, a subject as described herein is being treated with or has been previously treated with radiation therapy, chemotherapy, transplantation, immunotherapy, hormone therapy, or photodynamic therapy, or has been surgically treated.

In one embodiment, the present invention provides the use of a therapeutically acceptable amount of one or more compounds or compositions as described herein for treating, suppressing or inhibiting a proliferative disease in a subject. In another embodiment, the present invention provides the use of a therapeutically effective amount of one or more compounds or compositions as described herein for treating, suppressing or inhibiting a proliferative disease in a subject. In another embodiment, the present invention provides the use of a synergistically effective amount of one or more compounds or compositions as described herein for treating, suppressing or inhibiting a proliferative disease in a subject. In another embodiment, the present invention provides the use of a synergistically therapeutically effective amount of one or more compounds or compositions as described herein for treating, suppressing or inhibiting a proliferative disease in a subject.

In one embodiment, the proliferative disease comprises a fibromatous proliferative disease. In one embodiment, the proliferative disease comprises a benign fibrous tissue proliferation. In another embodiment, the proliferative disease comprises a fibrosarcoma.

In one embodiment, the proliferative disease comprises a pre-cancerous condition or a benign proliferative disorder.

In one embodiment, the term “pre-cancerous” or, alternatively, “pre-malignant” as used herein interchangeably refers to diseases, syndromes or other conditions associated with an increased risk of cancer. Pre-cancerous conditions in the context of the present invention include, but are not limited to: breast calcifications, vaginal intra-epithelial neoplasia, Barrett's esophagus, atrophic gastritis, dyskeratosis congenital, sideropenic dysphagia, lichen planus, oral submucous fibrosis, actinic keratosis, solar elastosis, cervical dysplasia, leukoplakia and erythroplakia.

In one embodiment, the term “benign hyperproliferative disorder” as used herein refers to a condition in which there is an abnormal growth and differentiation of cells and an increase in the amount of organic tissue that results from cell proliferation. The benign hyperproliferative disorder may be attributed to lack of response or inappropriate response to regulating factors, or alternatively to dysfunctional regulating factors.

In another embodiment, the hyperproliferative disorder comprises one or more of the hyperproliferative disorders described in WO 2019/222231, which is incorporated by reference herein in its entirety.

### Bisfluoroalkyl-1,4-benzodiazepinone Compounds

In some embodiments, compositions of the present invention or for use in the methods of the present invention comprise one or more gamma secretase inhibitors, one or more Notch inhibitors, or a combination thereof. In some embodiments, the gamma secretase inhibitor is a bisfluoroalkyl-1,4-benzodiazepinone compound. In some embodiments, the Notch inhibitor is a bisfluoroalkyl-1,4-benzodiazepinone compound.

In one embodiment, the present invention provides compositions comprising one or more compounds represented by the structure of Formula (I). In one embodiment, the composition comprises compounds represented by the structure of Formula (I):

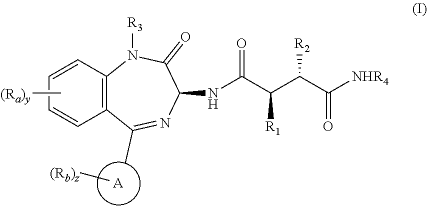

or a salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

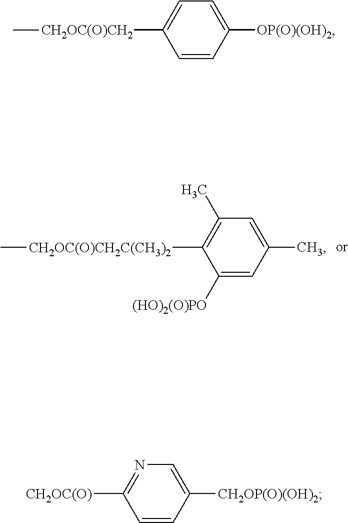

R_{y} is: —SCH₂CH(NH₂)C(O)OH, —SCH₂CH(NH₂)C(O)OCH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2.

In one embodiment, the present invention provides compositions comprising one or more compounds represented by the structure of Formula (II):

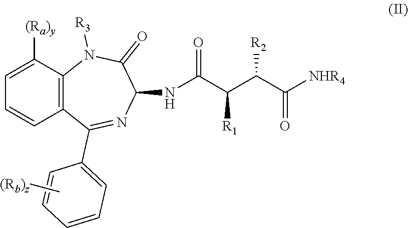

wherein R₃ is H or —CH₃; and y is zero or 1.

In one embodiment, the present invention provides compositions comprising one or more compounds represented by the structure of Formula (III). In one embodiment, the first composition comprises compounds of Formula (III):

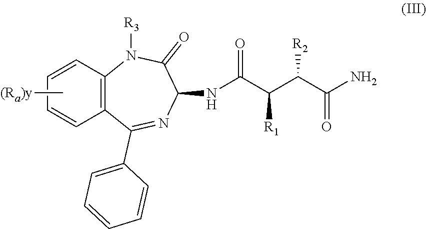

or prodrugs or salts thereof; wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H or —CH₃;
each Rₐ is independently F, Cl, —CN, —OCH₃, and/or —NHCH₂CH₂OCH₃; and
y is zero, 1, or 2.

In one embodiment, R₁ is —CH₂CF₃ or —CH₂CH₂CF₃ and R₂ is —CH₂CF₃ or —CH₂CH₂CF₃. In another embodiment, R₁ is —CH₂CH₂CF₃ and R₂ is —CH₂CH₂CF₃. In one embodiment, y is 1 or 2. In another embodiment, y is zero or 1. In one embodiment, y is zero.

In one embodiment, the compound of Formula (III) comprises: (2R,3S)—N-((3S)-1-methyl-2-oxo-5-phenyl-2,3-dihydro-1H-1,4-benzodiazepin-3-yl)-2,3-bis(3,3,3-trifluoropropyl)succinamide (Compound (1)):

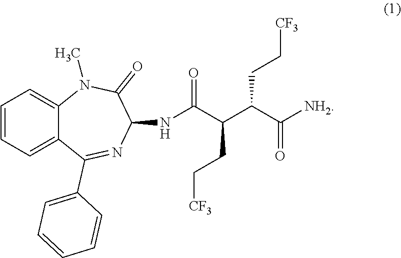 [SMILES: CN1C(=O)[C@@H](NC(=O)[C@H](CCC(F)(F)F)[C@H](CCC(F)(F)F)C(N)=O)N=C(c2ccccc2)c2ccccc21]

In another embodiment, the compound of Formula (III) comprises: (2R,3S)—N-((3S)-2-oxo-5-phenyl-2,3-dihydro-1H-1,4-benzodiazepin-3-yl)-2,3-bis(3,3,3-trifluoropropyl)succinamide (2):

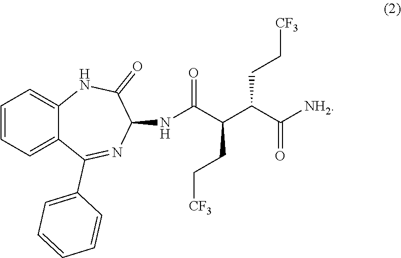 [SMILES: NC(=O)[C@@H](CCC(F)(F)F)[C@@H](CCC(F)(F)F)C(=O)N[C@H]1N=C(c2ccccc2)c2ccccc2NC1=O]

In another embodiment, the compound of Formula (III) comprises: (2R,3S)—N-((3S)-1-methyl-2-oxo-5-phenyl-2,3-dihydro-1H-1,4-benzodiazepin-3-yl)-2-(2,2,2-trifluoroethyl)-3-(3,3,3-trifluoropropyl)succinamide (3):

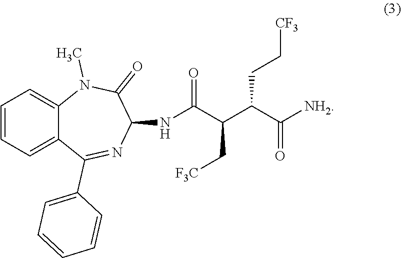 [SMILES: CN1C(=O)[C@@H](NC(=O)[C@H](CC(F)(F)F)[C@H](CCC(F)(F)F)C(N)=O)N=C(c2ccccc2)c2ccccc21]

In another embodiment, the compound of Formula (III) comprises: (2R,3S)—N-((3S)-1-methyl-2-oxo-5-phenyl-2,3-dihydro-1H-1,4-benzodiazepin-3-yl)-3-(2,2,2-trifluoroethyl)-2-(3,3,3-trifluoropropyl)succinamide (4):

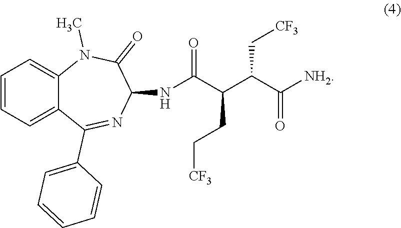 [SMILES: CN1C(=O)[C@@H](NC(=O)[C@H](CCC(F)(F)F)[C@H](CC(F)(F)F)C(N)=O)N=C(c2ccccc2)c2ccccc21]

In another embodiment, the compound of Formula (III) comprises: (2R,3S)—N-((3S)-1-(²H₃)methyl-2-oxo-5-phenyl-2,3-dihydro-1H-1,4-benzodiazepin-3-yl)-2,3-bis(3,3,3-trifluoropropyl)succinamide (5):

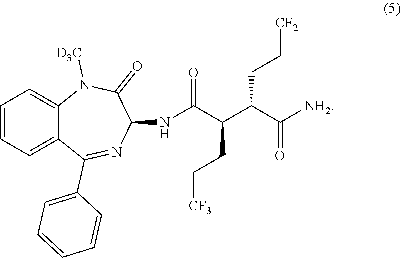

In another embodiment, the compound of Formula (III) comprises a compound of Formula (VI):

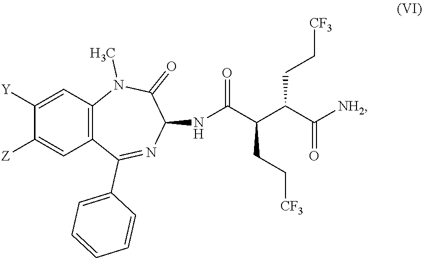

which, in one embodiment, comprises
(2R,3S)—N-((3S)-7-chloro-1-methyl-2-oxo-5-phenyl-2,3-dihydro-1H-1,4-benzodiazepin-3-yl)-2,3-bis(3,3,3-trifluoropropyl)succinamide (6), i.e. Y=H and Z=Cl; (2R,3S)—N-((3S)-8-methoxy-1-methyl-2-oxo-5-phenyl-2,3-dihydro-1H-1,4-benzodiazepin-3-yl)-2,3-bis(3,3,3-trifluoropropyl)succinamide (7), i.e. Y=OCH₃ and Z=H; (2R,3S)—N-((3S)-8-fluoro-1-methyl-2-oxo-5-phenyl-2,3-dihydro-1H-1,4-benzodiazepin-3-yl)-2,3-bis(3,3,3-trifluoropropyl)succinamide (8), i.e. Y=F and Z=H; (2R,3S)—N-((3S)-7-methoxy-1-methyl-2-oxo-5-phenyl-2,3-dihydro-1H-1,4-benzodiazepin-3-yl)-2,3-bis(3,3,3-trifluoropropyl)succinamide (9), Y=H and Z=OCH₃; (2R,3S)—N-((3S)-7-fluoro-1-methyl-2-oxo-5-phenyl-2,3-dihydro-1H-1,4-benzodiazepin-3-yl)-2,3-bis(3,3,3-trifluoropropyl)succinamide (10), i.e. Y=H and Z=F; or (2R,3S)—N-((3S)-8-chloro-1-methyl-2-oxo-5-phenyl-2,3-dihydro-1H-1,4-benzodiazepin-3-yl)-2,3-bis(3,3,3-trifluoropropyl)succinamide (11), i.e. Y=Cl and Z=H.

In another embodiment, the compound of Formula (III) comprises a compound of Formula (VII):

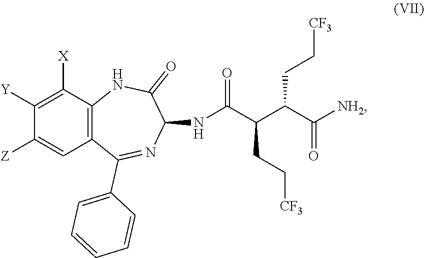

which in one embodiment, comprises
(2R,3S)—N-((3S)-9-methoxy-2-oxo-5-phenyl-2,3-dihydro-1H-1,4-benzodiazepin-3-yl)-2,3-bis(3,3,3-trifluoropropyl)succinamide (12), i.e. X=OCH₃, Y=H and Z=H; (2R,3S)—N-((3S)-8-methoxy-2-oxo-5-phenyl-2,3-dihydro-1H-1,4-benzodiazepin-3-yl)-2,3-bis(3,3,3-trifluoropropyl)succinamide (13), i.e. X=H, Y=OCH₃ and Z=H; (2R,3S)—N-((3S)-7-methoxy-2-oxo-5-phenyl-2,3-dihydro-1H-1,4-benzodiazepin-3-yl)-2,3-bis(3,3,3-trifluoropropyl)succinamide (14), i.e. X=H, Y=H and Z=OCH₃; (2R,3S)—N-((3S)-8-cyano-9-methoxy-2-oxo-5-phenyl-2,3-dihydro-1H-1,4-benzodiazepin-3-yl)-2,3-bis(3,3,3-trifluoropropyl)succinamide (15), i.e. X=OCH₃, Y=CN and Z=H; (2R,3S)—N-((3S)-8,9-dichloro-2-oxo-5-phenyl-2,3-dihydro-1H-1,4-benzodiazepin-3-yl)-2,3-bis(3,3,3-trifluoropropyl)succinamide (16), i.e. X=Cl, Y=Cl and Z=H; (2R,3S)—N-((3S)-9-fluoro-2-oxo-5-phenyl-2,3-dihydro-1H-1,4-benzodiazepin-3-yl)-2,3-bis(3,3,3-trifluoropropyl)succinamide (17), i.e. X=F, Y=H and Z=H; or (2R,3S)—N-((3S)-9-chloro-2-oxo-5-phenyl-2,3-dihydro-1H-1,4-benzodiazepin-3-yl)-2,3-bis(3,3,3-trifluoropropyl)succinamide (18), i.e. X=Cl, Y=H and Z=H.

In another embodiment, the compound of Formula (III) comprises: (2R,3S)—N-((3S)-2-oxo-5-phenyl-2,3-dihydro-1H-1,4-benzodiazepin-3-yl)-3-(4,4,4-trifluorobutyl)-2-(3,3,3-trifluoropropyl)succinamide (19);

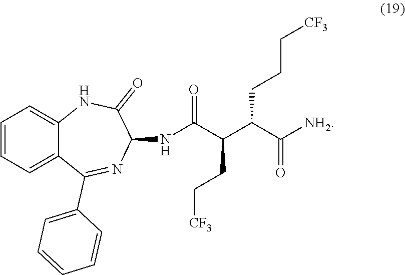 [SMILES: NC(=O)[C@@H](CCCC(F)(F)F)[C@@H](CCC(F)(F)F)C(=O)N[C@H]1N=C(c2ccccc2)c2ccccc2NC1=O]

In another embodiment, the compound of Formula (III) comprises: (2R,3S)—N-((3S)-8-methoxy-2-oxo-5-phenyl-2,3-dihydro-1H-1,4-benzodiazepin-3-yl)-3-(4,4,4-trifluorobutyl)-2-(3,3,3-trifluoropropyl)succinamide (20)

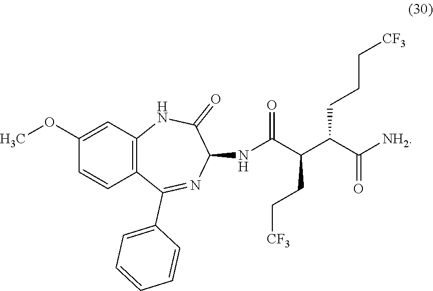 [SMILES: COc1ccc2c(c1)NC(=O)[C@@H](NC(=O)[C@H](CCC(F)(F)F)[C@H](CCCC(F)(F)F)C(N)=O)N=C2c1ccccc1]

In another embodiment, the compound of Formula (III) comprises: (2R,3S)—N-((3S)-9-((2-methoxyethyl)amino)-2-oxo-5-phenyl-2,3-dihydro-1H-1,4-benzodiazepin-3-yl)-2,3-bis(3,3,3-trifluoropropyl)succinamide (21)

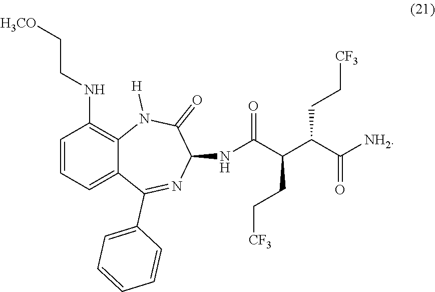 [SMILES: COCCNc1cccc2c1NC(=O)[C@@H](NC(=O)[C@H](CCC(F)(F)F)[C@H](CCC(F)(F)F)C(N)=O)N=C2c1ccccc1]

In another embodiment, the present invention provides compositions comprising one or more compounds represented by the structure of Formula (I):

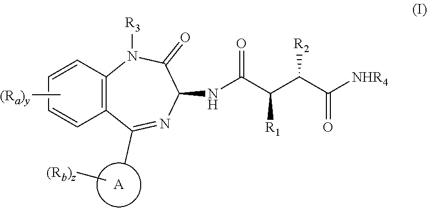

or a salt thereof, wherein:
R₁ is —CH₂CF₃;
R₂ is —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

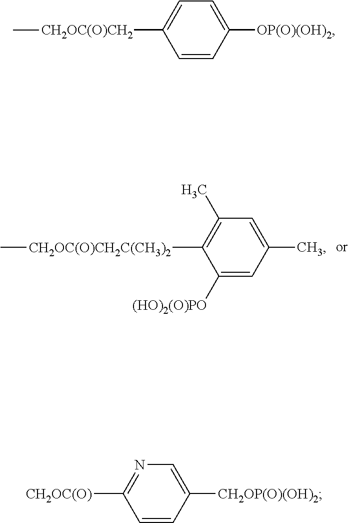

R_{y} is: —SCH₂CH(NH₂)C(O)OH, —SCH₂CH(NH₂)C(O)OCH₃,
or —SCH₂CH(NH₂)C(O)OC(CH₃)₃;
Ring A is phenyl or pyridinyl;
each Rₐ is independently Cl, C₁₋₃ alkyl, —CH₂OH, —CF₃, cyclopropyl, —OCH₃, and/or —O(cyclopropyl);
each R_{b} is independently F, Cl, —CH₃, —CH₂OH, —CF₃, cyclopropyl, and/or —OCH₃;
y is zero, 1 or 2; and
z is 1 or 2.

In another embodiment, Ring A is phenyl; and R₃ is H. In another embodiment, R₂ is —CH₂CH₂CF₃; and Ring A is phenyl. In another embodiment, R₂ is —CH₂CH₂CF₃; Ring A is phenyl; Rₐ is C₁₋₃ alkyl or —CH₂OH; each R_{b} is independently F and/or Cl; and y is 1.

In another embodiment, the present invention provides compositions comprising one or more compounds represented by the structure of Formula (IV):

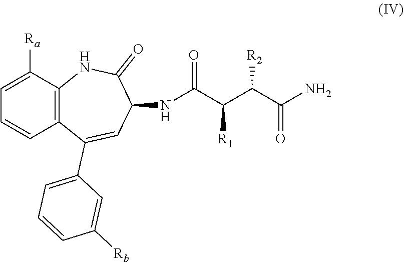

In another embodiment, the present invention provides compositions comprising one or more compounds represented by the structure of Formula (V):

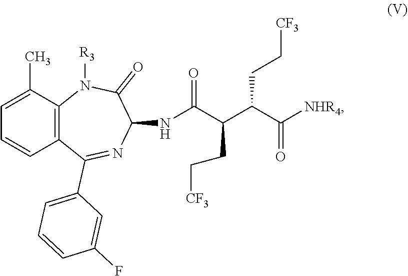

wherein R₃ is H or Rₓ.
   In another embodiment, the present invention provides compositions comprising (2R,3S)—N-((3S)-5-(3-fluorophenyl)-9-methyl-2-oxo-2,3-dihydro-1H-1,4-benzodiazepin-3-yl)-2,3-bis(3,3,3-trifluoropropyl)succinamide (22); (2R,3S)—N-((3S)-5-(3-chlorophenyl)-9-ethyl-2-oxo-2,3-dihydro-1H-1,4-benzodiazepin-3-yl)-2,3-bis(3,3,3-trifluoropropyl)succinamide (23); (2R,3S)—N-((3S)-5-(3-chlorophenyl)-9-isopropyl-2-oxo-2,3-dihydro-1H-1,4-benzodiazepin-3-yl)-2,3-bis(3,3,3-trifluoropropyl)succinamide (24); (2R,3S)—N-(9-chloro-5-(3,4-dimethylphenyl)-2-oxo-2,3-dihydro-1H-1,4-benzodiazepin-3-yl)-3-(4,4,4-trifluorobutyl)-2-(3,3,3-trifluoropropyl)succinamide (25); (2R,3S)—N-(9-chloro-5-(3,5-dimethylphenyl)-2-oxo-2,3-dihydro-1H-1,4-benzodiazepin-3-yl)-3-(4,4,4-trifluorobutyl)-2-(3,3,3-trifluoropropyl)succinamide (26); (2R,3S)—N-((3S)-9-ethyl-5-(3-methylphenyl)-2-oxo-2,3-dihydro-1H-1,4-benzodiazepin-3-yl)-2,3-bis(3,3,3-trifluoropropyl)succinamide (27); (2R,3S)—N-((3S)-5-(3-chlorophenyl)-9-methyl-2-oxo-2,3-dihydro-1H-1,4-benzodiazepin-3-yl)-2,3-bis(3,3,3-trifluoropropyl)succinamide (28); (2R,3S)—N-((3S)-5-(3-chlorophenyl)-9-methyl-2-oxo-2,3-dihydro-1H-1,4-benzodiazepin-3-yl)-3-(4,4,4-trifluorobutyl)-2-(3,3,3-trifluoropropyl)succinamide (29); (2R,3S)—N-((3S)-5-(3-methylphenyl)-2-oxo-9-(trifluoromethyl)-2,3-dihydro-1H-1,4-benzodiazepin-3-yl)-2,3-bis(3,3,3-trifluoropropyl)succinamide (30); (2R,3S)—N-((3S)-9-chloro-5-(3,5-dimethylphenyl)-2-oxo-2,3-dihydro-1H-1,4-benzodiazepin-3-yl)-2,3-bis(3,3,3-trifluoropropyl)succinamide (31); (2R,3S)—N-((3S)-5-(3-methylphenyl)-2-oxo-9-(trifluoromethyl)-2,3-dihydro-1H-1,4-benzodiazepin-3-yl)-3-(4,4,4-trifluorobutyl)-2-(3,3,3-trifluoropropyl)succinamide (32); (2R,3S)—N-((3S)-9-isopropyl-5-(3-methylphenyl)-2-oxo-2,3-dihydro-1H-1,4-benzodiazepin-3-yl)-2,3-bis(3,3,3-trifluoropropyl)succinamide (33); (2R,3S)—N-((3S)-9-(cyclopropyloxy)-5-(3-methylphenyl)-2-oxo-2,3-dihydro-1H-1,4-benzodiazepin-3-yl)-3-(4,4,4-trifluorobutyl)-2-(3,3,3-trifluoropropyl)succinamide (34); (2R,3S)—N-((3S)-9-(cyclopropyloxy)-5-(3-methylphenyl)-2-oxo-2,3-dihydro-1H-1,4-benzodiazepin-3-yl)-2,3-bis(3,3,3-trifluoropropyl)succinamide (35); (2R,3S)—N-((3S)-9-chloro-5-(3-methylphenyl)-2-oxo-2,3-dihydro-1H-1,4-benzodiazepin-3-yl)-3-(4,4,4-trifluorobutyl)-2-(3,3,3-trifluoropropyl) succinamide (36); (2R,3S)—N-((3S)-9-methyl-2-oxo-5-(3-(trifluoromethyl)phenyl)-2,3-dihydro-1H-1,4-benzodiazepin-3-yl)-3-(4,4,4-trifluorobutyl)-2-(3,3,3-trifluoropropyl) succinamide (37); (2R,3S)—N-((3S)-9-methyl-2-oxo-5-(3-(trifluoromethyl) phenyl)-2,3-dihydro-1H-1,4-benzodiazepin-3-yl)-2,3-bis(3,3,3-trifluoropropyl) succinamide (38); (2R,3S)—N-((3S)-9-chloro-5-(2-methylphenyl)-2-oxo-2,3-dihydro-1H-1,4-benzodiazepin-3-yl)-2,3-bis(3,3,3-trifluoropropyl)succinamide (39); (2R,3S)—N-((3S)-5-(4-fluorophenyl)-9-methyl-2-oxo-2,3-dihydro-1H-1,4-benzodiazepin-3-yl)-2,3-bis(3,3,3-trifluoropropyl)succinamide (40); (2R,3S)—N-((3S)-9-chloro-5-(3-cyclopropylphenyl)-2-oxo-2,3-dihydro-1H-1,4-benzodiazepin-3-yl)-2,3-bis(3,3,3-trifluoropropyl)succinamide (41); (2R,3S)—N-((3S)-5-(3-chlorophenyl)-9-methoxy-2-oxo-2,3-dihydro-1H-1,4-benzodiazepin-3-yl)-2,3-bis(3,3,3-trifluoropropyl)succinamide (42); (2R,3S)—N-((3S)-5-(4-chlorophenyl)-9-methoxy-2-oxo-2,3-dihydro-1H-1,4-benzodiazepin-3-yl)-2,3-bis(3,3,3-trifluoropropyl)succinamide (43); (2R,3S)—N-((3S)-9-chloro-5-(3-methylphenyl)-2-oxo-2,3-dihydro-1H-1,4-benzodiazepin-3-yl)-2,3-bis(3,3,3-trifluoropropyl)succinamide (44); (2R,3S)—N-((3S)-5-(3-methylphenyl)-9-methoxy-2-oxo-2,3-dihydro-1H-1,4-benzodiazepin-3-yl)-2,3-bis(3,3,3-trifluoropropyl)succinamide (45); (2R,3S)—N-((3S)-5-(4-(hydroxymethyl)phenyl)-2-oxo-2,3-dihydro-1H-1,4-benzodiazepin-3-yl)-2,3-bis(3,3,3-trifluoropropyl)succinamide (46); (2R,3S)—N-((3S)-5-(2-methylphenyl)-2-oxo-2,3-dihydro-1H-1,4-benzodiazepin-3-yl)-2,3-bis(3,3,3-trifluoropropyl)succinamide (47); (2R,3S)—N-((3S)-5-(3-methylphenyl)-2-oxo-2,3-dihydro-1H-1,4-benzodiazepin-3-yl)-2,3-bis(3,3,3-trifluoropropyl)succinamide (48); (2R,3S)—N-((3S)-9-methoxy-2-oxo-5-(5-(trifluoromethyl)-2-pyridinyl)-2,3-dihydro-1H-1,4-benzodiazepin-3-yl)-2,3-bis(3,3,3-trifluoropropyl)succinamide (49); (2R,3S)—N-((3S)-5-(5-chloro-2-pyridinyl)-9-methoxy-2-oxo-2,3-dihydro-1H-1,4-benzodiazepin-3-yl)-2,3-bis(3,3,3-trifluoropropyl)succinamide (50); (2R,3S)—N-((3S)-5-(4-methoxyphenyl)-2-oxo-2,3-dihydro-1H-1,4-benzodiazepin-3-yl)-2,3-bis(3,3,3-trifluoropropyl)succinamide (51); (2R,3S)—N-((3S)-5-(4-methylphenyl)-2-oxo-2,3-dihydro-1H-1,4-benzodiazepin-3-yl)-2,3-bis(3,3,3-trifluoropropyl)succinamide (52); (2R,3S)—N-((3S)-5-(3-fluorophenyl)-9-(hydroxymethyl)-2-oxo-2,3-dihydro-1H-1,4-benzodiazepin-3-yl)-2,3-bis(3,3,3-trifluoropropyl)succinamide (53); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valinate (54); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-alaninate (55); S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteine (56); tert-butyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteinate (57); methyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl) hexanoyl)amino)-L-cysteinate (58); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl (4-(phosphonooxy)phenyl)acetate (59); and ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valyl-L-valinate (60); and salts thereof.

In another embodiment, the present invention provides compositions comprising one or more compounds represented by the structure of Formula (I):

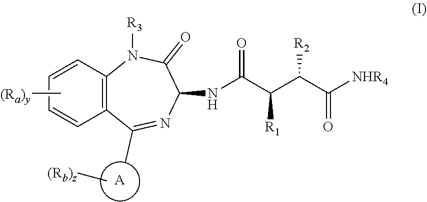

or a salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

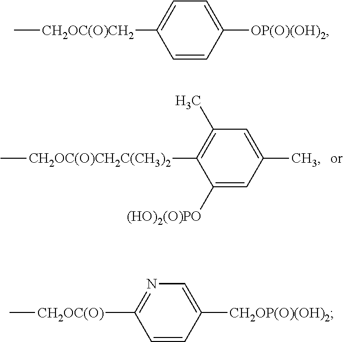

R_{y} is: —SCH₂CH(NH₂)C(O)OH, —SCH₂CH(NH₂)C(O)OCH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is zero, 1, or 2
provided that if Ring A is phenyl, z is zero, and y is 1 or 2 then at least one Rₐ is C₁₋₃ alkyl, —CH₂OH, —CF₃, cyclopropyl, or —O(cyclopropyl);
provided that if R₃ is Rₓ then R₄ is H; and
provided that if R₄ is R_{y} then R₃ is H or —CH₃.

In another embodiment, the structure as described hereinabove comprises one or more of the following provisos: provided that if Ring A is phenyl, z is zero, and y is 1 or 2 then at least one Rₐ is C₁₋₃ alkyl, —CH₂OH, —CF₃, cyclopropyl, or —O(cyclopropyl); provided that if R₃ is Rₓ then R₄ is H; and provided that if R₄ is R_{y} then R₃ is H or —CH₃.

In another embodiment, the present invention provides compositions comprising one or more compounds represented by the following structure:

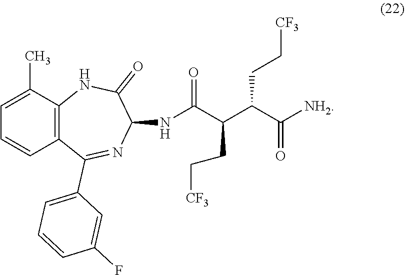 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

In another embodiment, the compounds as described herein comprise prodrugs of one or more of the compounds.

U.S. Pat. No. 9,273,014, which is incorporated by reference herein in its entirety, discloses various compounds of Formula (I):

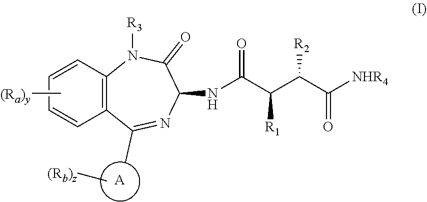

or a salt thereof, wherein:
R₁ is —CH₂CH₂CF₃;
R₂ is —CH₂CH₂CF₃ or —CH₂CH₂CH₂CF₃;
R₃ is H, —CH₃, or Rₓ;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂,

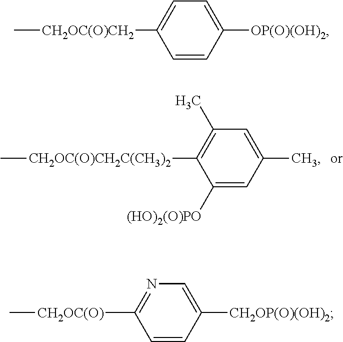

R_{y} is: —SCH₂CH(NH₂)C(O)OH, —SCH₂CH(NH₂)C(O)OCH₃,
or —SCH₂CH(NH₂)C(O)OC(CH₃)₃;
Ring A is phenyl or pyridinyl;
each Rₐ is independently Cl, C₁₋₃ alkyl, —CH₂OH, —CF₃, cyclopropyl, —OCH₃, and/or —O(cyclopropyl);
each R_{b} is independently F, Cl, —CH₃, —CH₂OH, —CF₃, cyclopropyl, and/or —OCH₃;
y is zero, 1, or 2; and
z is 1 or 2.

U.S. Pat. No. 9,273,014 also discloses the compound of Formula (22):

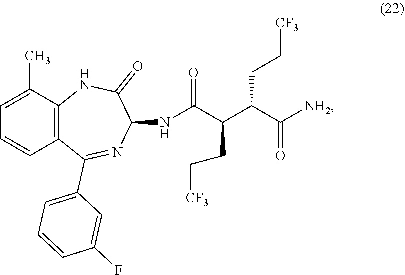 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

which, in one embodiment, has the chemical name (2R,3S)—N-((3S)-5-(3-fluorophenyl)-9-methyl-2-oxo-2,3-dihydro-1H-1,4-benzodiazepin-3-yl)-2,3-bis(3,3,3-trifluoropropyl)succinamide. U.S. Pat. No. 9,273,014 also discloses a process for synthesizing the compounds as well as other compounds of Formula (I), which are to be considered as part of the present invention.

U.S. Pat. No. 8,629,136, which is incorporated by reference herein in its entirety, discloses compounds of Formula (III):

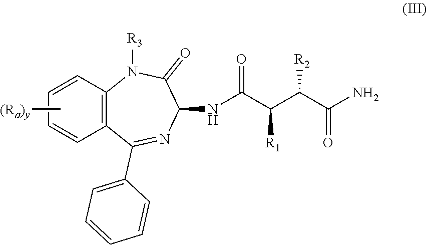

or a salt thereof, wherein:
R₃ is H or —CH₃; and
each Rₐ is independently F, Cl, —CN, —OCH₃ and/or —NHCH₂CH₂OCH₃.
   U.S. Pat. No. 8,629,136 also discloses the compound of Formula (1):

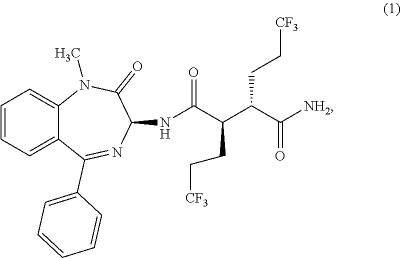 [SMILES: CN1C(=O)[C@@H](NC(=O)[C@H](CCC(F)(F)F)[C@H](CCC(F)(F)F)C(N)=O)N=C(c2ccccc2)c2ccccc21]

which, in one embodiment, has the chemical name (2R,3S)—N-((3S)-1-methyl-2-oxo-5-phenyl-2,3-dihydro-1H-1,4-benzodiazepin-3-yl)-2,3-bis(3,3,3-trifluoropropyl)succinamide. In one embodiment, the compounds are Notch inhibitors. U.S. Pat. No. 8,629,136 discloses a process for synthesizing the compounds as well as other compounds of Formula (I), which are to be considered as part of the present invention.

The present invention may be embodied in other specific forms without departing from the spirit or essential attributes thereof. This invention encompasses all combinations of the aspects and/or embodiments of the invention noted herein. It is understood that any and all embodiments of the present invention may be taken in conjunction with any other embodiment or embodiments to describe addition more embodiments. It is also to be understood that each individual element of the embodiments is meant to be combined with any and all other elements from any embodiment to describe an additional embodiment.

### Combined Treatments/Therapies

In one embodiment, the present invention provides combination therapies comprising a first composition comprising one or more compounds represented by the structure of Formula (I) as described herein and a second composition comprising one or more therapeutic agents. In one embodiment, the therapeutic agent comprises a chemotherapeutic agent.

In some embodiments, the term “chemotherapeutic agent” may encompass any chemical agent that has therapeutic utility in the treatment of hyperproliferative disorders, such as cancer.

In another embodiment, the present invention provides a composition comprising two or three or more compounds. In other embodiments, the present invention provides a combination treatment comprising two or three or more compounds. In one embodiment, the first compound comprises one or more compounds represented by the structure of Formula (I) as described herein.

In some embodiments, the second composition or additional composition comprises an HDAC inhibitor, a Bcl2 inhibitor, a CDK 4/6 inhibitor, a tyrosine kinase inhibitor, a FGFR inhibitor, an inhibitor of PRMT5, an inhibitor of Bromodomain and BET, a retinoic acid, or a combination thereof. In other embodiments, the one or more therapeutic agents comprises panobinostat, romidepsin, vorinostat, belinostat, venetoclax, lisaftoclax, (APG-2575), palbociclib, ribociclib, abemaciclib, pazopanib, sorafenib, lenvatinib, regorafenib, rivoceranib (apatinib), erdafitinib, pemigatinib, or infigratinib.

In other embodiments, the second composition or additional composition comprises an inhibitor of a peptide or protein comprising one or more B-cell leukemia/lymphoma-2 (Bcl-2) homology [BH] motifs. In some embodiments, the second compound comprises an inhibitor of a Bcl-2 family member. In one embodiment, the second composition comprises one or more Bcl-2 inhibitors, as described in PCT/US2022/028956, which is incorporated herein by reference in its entirety, or Bcl-2 inhibitors that are known in the art.

In another embodiment, any of the compositions or combinations as described herein for any of the uses as described herein may be used in combination with other anti-cancer treatments as described herein or as described in PCT Publication No. WO 2019/222231, which is incorporated by reference herein in its entirety, or anti-cancer treatments that are known in the art.

In some embodiments, the present invention provides a first composition comprising one or more compounds represented by the structure of Formula (I) as described herein and an additional composition comprising one or more therapeutic agents.

In some embodiments, the present invention provides a composition comprising one or more compounds represented by the structure of Formula (I) as described herein and an additional composition comprising one or more HDAC inhibitors. In one embodiment, the HDAC inhibitor comprises Panobinostat. In another embodiment, the HDAC inhibitor comprises Romidepsin. In another embodiment, the HDAC inhibitor comprises Vorinostat. In another embodiment, the HDAC inhibitor comprises Belinostat.

In some embodiments, the present invention provides a composition comprising one or more compounds represented by the structure of Formula (I) as described herein and an additional composition comprising one or more CDK 4/6 inhibitors. In one embodiment, the CDK 4/6 inhibitor comprises Palbociclib. In another embodiment, the CDK 4/6 inhibitor comprises ribociclib. In another embodiment, the CDK 4/6 inhibitor comprises abemaciclib. In another embodiment, the CDK 4/6 inhibitor comprises lerociclib or trilaciclib.

In some embodiments, the present invention provides a first composition comprising one or more compounds represented by the structure of Formula (I) as described herein and an additional composition comprising one or more TKIs. In one embodiment, the TKI comprises pazopanib. In another embodiment, the TKI comprises sorafenib. In another embodiment, the TKI comprises lenvatinib. In another embodiment, the TKI comprises regorafenib. In another embodiment, the TKI comprises lenvatinib. In another embodiment, the TKI comprises rivoceranib (apatinib). In another embodiment, the TKI comprises Lapatinib.

In some embodiments, the present invention provides a first composition comprising one or more compounds represented by the structure of Formula (I) as described herein and an additional composition comprising one or more fibroblast growth factor receptor (FGFR) inhibitors. In one embodiment, the FGFR inhibitor comprises JNJ-42756493 (erdafitinib). In another embodiment, the FGFR inhibitor comprises pemigatinib. In another embodiment, the FGFR inhibitor comprises infigratinib.

In one embodiment, the FGFR inhibitor comprises an irreversible inhibitor. In one embodiment, the FGFR inhibitor comprises FIIN-2/FIIN-3, PRN1371, RLY-4008, derazantinib, AZD4547, futibatinib, CH5183284, or a combination thereof. In another embodiment, the FGFR inhibitor comprises brivanib, dovitinib, ponatinib, or a combination thereof. In another embodiment, the FGFR inhibitor comprises, Ly2874455, NVP-BGJ398, INCB054828, TAS-120, BLU-554, H3B-6527, FGF401, or a combination thereof.

In some embodiments, the additional composition comprises one or more inhibitors of protein arginine methyltransferase 5 (PRMT5). In some embodiments, the PRMT5 comprises GSK3326595 (EPZ015938). In some embodiments, the PRMT5 comprises GSK3235025 (EPZ015666). In some embodiments, the PRMT5 comprises GSK3368715 (EPZ019997). In some embodiments, the PRMT5 comprises JNJ-64619178.

In some embodiments, the additional composition comprises one or more inhibitors of Bromodomain and Extra-Terminal motif (BET). In some embodiments, the BET inhibitor comprises I-BET 151 (GSK1210151A), I-BET 762 (GSK525762), OTX-015, TEN-010, CPI-203, CPI-0610, olinone, RVX-208, ABBV-744, LY294002, AZD5153, MT-1, or a combination thereof.

In some embodiments, the additional composition comprises one or more Histone deacetylase inhibitors (HDIs). In some embodiments, the HDI comprises hydroxamic acids (or hydroxamate), which, in one embodiment, comprises trichostatin A. In some embodiments, the HDI comprises cyclic tetrapeptides, which, in one embodiment, comprises trapoxin B. In some embodiments, the HDI comprises depsipeptide. In some embodiments, the HDI comprises benzamide. In some embodiments, the HDI comprises an electrophilic ketone. In some embodiments, the HDI comprises aliphatic acids, which, in one embodiment, comprises phenylbutyrate or valproic acid. In some embodiments, the HDI comprises hydroxamic acids, which, in some embodiments, comprise vorinostat (SAHA), belinostat (PXD101), LAQ824, panobinostat (LBH589), or a combination thereof. In some embodiments, the HDI comprises a benzamide. In some embodiments, the HDI comprises entinostat (MS-275), tacedinaline (CI994), and mocetinostat (MGCD0103), or a combination thereof. In some embodiments, the HDI comprises nicotinamide. In some embodiments, the HDI comprises Nicotinamide adenine dinucleotide (NAD) derivatives, which, in some embodiments, comprise dihydrocoumarin, naphthopyranone, 2-hydroxynaphthaldehydes, or a combination thereof.

In some embodiments, the additional composition comprises one or more Tyrosine Kinase Inhibitors (TKIs). In some embodiments, the TKI comprises Apatinib (Rivoceranib; YN968D1). In some embodiments, the TKI comprises Lapatinib. In some embodiments, the TKI comprises Lenvatinib. In some embodiments, the TKI comprises Lenvatinib. In some embodiments, the TKI comprises Apatinib. In some embodiments, the TKI comprises Lapatinib.

In some embodiments, the additional composition comprises retinoic acid. In some embodiments, the retinoic acid comprises all-trans retinoic acid (ATRA) (tretin-X, tretinoin). In some embodiments, the retinoic acid comprises alitretinoin. In some embodiments, the retinoic acid comprises isotretinoin.

In some embodiments, the additional composition comprises at least one anti-cancer agent. In some embodiments, the additional composition comprises one anti-cancer agent. In some embodiments, the additional composition comprises two anti-cancer agents. In some embodiments, the additional composition comprises 3 anti-cancer agents. In some embodiments, the additional composition comprises 4 anti-cancer agents. In some embodiments, the additional composition comprises 5 anti-cancer agents. In some embodiments, the additional composition comprises 6 anti-cancer agents.

In another embodiment, the present invention provides a combination therapy comprising one or more compounds represented by the structure of Formula (I) or Formula (IV) as described herein and osimertinib (mereletinib; Tagrisso).

In another embodiment, the present invention provides a combination therapy comprising one or more compounds represented by the structure of Formula (I) or Formula (IV) as described herein and one or more chromatin modulators.

In another embodiment, the present invention provides a combination therapy comprising one or more compounds represented by the structure of Formula (I) or Formula (IV) as described herein and a compound or polypeptide that indirectly targets Myb/Myc.

In some embodiments, the combination therapy further comprises an agent for treating Desmoid tumors. Non-limiting examples of agents include nonsteroidal anti-inflammatory drugs, hormone therapy, targeted therapy, chemotherapy, or a combination thereof. In some embodiments, NSAIDs comprise celecoxib, ibuprofen, indomethacin, naproxen, and sulindac. In some embodiments, hormone therapy comprises Tamoxifen. In some embodiments, targeted therapy comprises one or more TKIs. In one embodiment, the TKI comprises sorafenib imatinib pazopanib

In some embodiments, chemotherapy comprises methotrexate, vinblastine, liposomal doxorubicin, doxorubicin, dacarbazine and doxorubicin, vinorelbine, or a combination thereof.

In one embodiment, a method is provided for treating cancer comprising administering to a mammal in need thereof a first composition comprising one or more compounds represented by the structure of Formula (I) or Formula (IV) as described herein and administering a second composition comprising one or more Bcl-2 family inhibitors.

In one embodiment, the phrase “chemotherapeutic agent” comprises: alkylating agents (including mustard, nitrogen mustards, methanesulphonate, busulphan, alkyl sulfonates, nitrosoureas, ethylenimine derivatives, and triazenes or combinations thereof); anti-angiogenics (including matrix metalloproteinase inhibitors); antimetabolites (including adenosine deaminase inhibitors, folic acid antagonists, purine analogues, and pyrimidine analogues); antibiotics or antibodies (including monoclonal antibodies, CTLA-4 antibodies, anthracyclines); aromatase inhibitors; cell-cycle response modifiers; enzymes; farnesyl-protein transferase inhibitors; hormonal and antihormonal agents and steroids (including synthetic analogs, glucocorticoids, estrogens/anti-estrogens [e.g., SERMs], androgens/anti-androgens, progestins, progesterone receptor agonists, and luteinizing hormone-releasing [LHRH] agonists and antagonists); insulin-like growth factor (IGF)/insulin-like growth factor receptor (IGFR) system modulators (including IGFR1 inhibitors); integrin-signaling inhibitors; kinase inhibitors (including multi-kinase inhibitors and/or inhibitors of Src kinase or Src/ab1, cyclin dependent kinase [CDK] inhibitors, panHer, Her-1 and Her-2 antibodies, VEGF inhibitors, including anti-VEGF antibodies, EGFR inhibitors, PARP (poly ADP-ribose polymerase) inhibitors, mitogen-activated protein [MAP] inhibitors, MET inhibitors, Aurora kinase inhibitors, PDGF inhibitors, and other tyrosine kinase inhibitors or serine/threonine kinase inhibitors; microtubule-disruptor agents, such as ecteinascidins or their analogs and derivatives; microtubule-stabilizing agents such as taxanes, Platinum-based antineoplastic drugs (platins) such as cisplatin, carboplatin, oxaliplatin, nedaplatin, triplatin tetranitrate, phenanthriplatin, picoplatin and satraplatin and the naturally-occurring epothilones and their synthetic and semi-synthetic analogs; microtubule-binding, destabilizing agents (including vinca alkaloids); topoisomerase inhibitors; prenyl-protein transferase inhibitors; platinum coordination complexes; signal transduction inhibitors; Histone deacetylase inhibitors (HDIs), inhibitors of Bromodomain and Extra-Terminal motif (BET), inhibitors of protein arginine methyltransferase 5 (PRMT5), retinoic acids and other agents used as anti-cancer and cytotoxic agents such as biological response modifiers, growth factors, and immune modulators.

Accordingly, the combination therapies of the present invention may be administered together with other anti-cancer treatments useful in the treatment of cancer or other proliferative diseases. The invention herein further comprises use of the first and second compositions of the present invention in preparing medicaments for the treatment of cancer.

The compounds, compositions, and combinations of the present invention can be formulated or co-administered with other therapeutic agents that are selected for their particular usefulness in addressing side effects associated with the aforementioned conditions. For example, compounds of the invention may be formulated with agents to prevent nausea, hypersensitivity and gastric irritation, such as antiemetics, and H₁ and H₂ antihistamines.

In one embodiment, pharmaceutical compositions are provided comprising a compound of Formula (I) or prodrug thereof, one or more additional agents selected from a kinase inhibitory agent (small molecule, polypeptide, and antibody), an immunosuppressant, an anti-cancer agent, an anti-viral agent, anti-inflammatory agent, antifungal agent, antibiotic, or an anti-vascular hyperproliferation compound; and any pharmaceutically acceptable carrier, adjuvant or vehicle. The chemotherapeutic agents, when employed in combination with the compounds of the present invention, may be used, for example, in those amounts indicated in the Physicians' Desk Reference (PDR) or as otherwise determined by one of ordinary skill in the art.

### Pharmaceutical Compositions

### Formulations

The present invention also provides a class of pharmaceutical compositions comprising any of the compounds of Formula (I) or Formula (IV), and optionally a second composition comprising one or more therapeutic agents, and one or more non-toxic, pharmaceutically acceptable carriers and/or diluents and/or adjuvants (collectively referred to herein as “carrier” materials) and, if desired, other active ingredients. In some embodiments, a pharmaceutical composition comprises a first composition, as described herein. In some embodiments, a pharmaceutical composition comprises a second composition, as described herein.

The compounds of Formula (I) or Formula (IV) may be administered by any suitable route, preferably in the form of a pharmaceutical composition adapted to such a route, and in a dose effective for the treatment intended. The compounds and pharmaceutical compositions of the present invention may, for example, be administered in dosage unit formulations containing conventional pharmaceutically acceptable carriers, adjuvants, and vehicles. For example, the pharmaceutical carrier may contain a mixture of mannitol or lactose and microcrystalline cellulose. The mixture may contain additional components such as a lubricating agent, e.g., magnesium stearate and a disintegrating agent such as crospovidone. The carrier mixture may be filled into a gelatin capsule or compressed as a tablet. The pharmaceutical composition may be administered as an oral dosage form or an infusion, for example.

For oral administration, the pharmaceutical composition may be in the form of, for example, a tablet, capsule, liquid capsule, suspension, or liquid. The pharmaceutical composition is preferably made in the form of a dosage unit containing a particular amount of the active ingredient. For example, the pharmaceutical composition may be provided as a tablet or capsule comprising an amount of active ingredient in the range of from about 1 to 2000 mg, preferably from about 1 to 500 mg, and more preferably from about 5 to 150 mg. A suitable daily dose for a human or other mammal may vary widely depending on the condition of the subject and other factors, but can be determined using routine methods.

Any pharmaceutical composition contemplated herein can, for example, be delivered orally via any acceptable and suitable oral preparations. Exemplary oral preparations, include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the invention can contain at least one agent selected from sweetening agents, flavoring agents, coloring agents, demulcents, antioxidants, and preserving agents.

A tablet can, for example, be prepared by admixing at least one compound of Formula (I) or Formula (IV) with at least one non-toxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium croscarmellose, corn starch, and alginic acid; binding agents, such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water-soluble taste masking materials, include, but are not limited to, hydroxypropyl-methylcellulose and hydroxypropyl-cellulose. Exemplary time delay materials, include, but are not limited to, ethyl cellulose and cellulose acetate butyrate.

Hard gelatin capsules can, for example, be prepared by mixing at least one compound of Formula (I) or Formula (IV) with at least one inert solid diluent, such as, for example, calcium carbonate; calcium phosphate; and kaolin.

Soft gelatin capsules can, for example, be prepared by mixing at least one compound of Formula (I) or Formula (IV) with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) or Formula (IV) with at least one excipient suitable for the manufacture of an aqueous suspension. Exemplary excipients suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example heptadecaethylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) or Formula (IV) in either a vegetable oil, such as, for example, arachis oil; olive oil; sesame oil; and coconut oil; or in mineral oil, such as, for example, liquid paraffin. An oily suspension can also contain at least one thickening agent, such as, for example, beeswax; hard paraffin; and cetyl alcohol. In order to provide a palatable oily suspension, at least one of the sweetening agents already described hereinabove, and/or at least one flavoring agent can be added to the oily suspension. An oily suspension can further contain at least one preservative, including, but not limited to, for example, an antioxidant, such as, for example, butylated hydroxyanisol, and alpha-tocopherol.

Dispersible powders and granules can, for example, be prepared by admixing at least one compound of Formula (I) or Formula (IV) with at least one dispersing and/or wetting agent; at least one suspending agent; and/or at least one preservative. Suitable dispersing agents, wetting agents, and suspending agents are as already described above. Exemplary preservatives include, but are not limited to, for example, anti-oxidants, e.g., ascorbic acid. In addition, dispersible powders and granules can also contain at least one excipient, including, but not limited to, for example, sweetening agents; flavoring agents; and coloring agents.

An emulsion of at least one compound of Formula (I) or Formula (IV) can, for example, be prepared as an oil-in-water emulsion. The oily phase of the emulsions comprising compounds of Formula (I) or Formula (IV) may be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase may comprise merely an emulsifier, it may comprise a mixture of at least one emulsifier with a fat or an oil or with both a fat and an oil. Suitable emulsifying agents include, but are not limited to, for example, naturally-occurring phosphatides, e.g., soy bean lecithin; esters or partial esters derived from fatty acids and hexitol anhydrides, such as, for example, sorbitan monooleate; and condensation products of partial esters with ethylene oxide, such as, for example, polyoxyethylene sorbitan monooleate. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. An emulsion can also contain a sweetening agent, a flavoring agent, a preservative, and/or an antioxidant. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

In another embodiment, the compounds of Formula (I) or Formula (IV) can be formulated as a nanoparticle, lipid nanoparticle, microparticle or liposome.

The compounds of Formula (I) or Formula (IV) can, for example, also be delivered intravenously, subcutaneously, and/or intramuscularly via any pharmaceutically acceptable and suitable injectable form. Exemplary injectable forms include, but are not limited to, for example, sterile aqueous solutions comprising acceptable vehicles and solvents, such as, for example, water, Ringer's solution, and isotonic sodium chloride solution; sterile oil-in-water microemulsions; and aqueous or oleaginous suspensions. For example, the first or second composition may be provided for intravenous administration comprising an amount of active ingredient in the range of from about 0.2 to 150 mg. In another embodiment, the active ingredient is present in the range of from about 0.3 to 10 mg. In another embodiment, the active ingredient is present in the range of from about 4 to 8.4 mg. In one embodiment, the active ingredient is administered at a dose of about 4 mg. In another embodiment, the active ingredient is administered at a dose of about 6 mg. In another embodiment, the active ingredient is administered at a dose of about 8.4 mg.

In another embodiment, the active ingredient is administered at a dose of about 0.3 mg. In another embodiment, the active ingredient is administered at a dose of about 0.6 mg. In another embodiment, the active ingredient is administered at a dose of about 1.2 mg. In another embodiment, the active ingredient is administered at a dose of about 2.4 mg. In another embodiment, the active ingredient is administered at a dose of about 4 mg. In another embodiment, the active ingredient is administered at a dose of about 6 mg. In another embodiment, the active ingredient is administered at a dose of about 8 mg.

In one embodiment, the first composition comprising one or more compounds represented by the structure of Formula (I) or Formula (IV) as described herein may be provided for intravenous administration. In one embodiment, the first composition for use as described herein comprises 0.2 to 150 mg of one or more compounds represented by the structure of Formula (I), as described herein. In another embodiment, the first composition for use as described herein comprises one or more compounds represented by the structure of Formula (I) in the range of from about 0.3 to 10 mg. In another embodiment, one or more compounds represented by the structure of Formula (I) is present in the range of from about 4 to 8.4 mg. In one embodiment, one or more compounds represented by the structure of Formula (I) is administered at a dose of about 2.4 mg. In another embodiment, one or more compounds represented by the structure of Formula (I) is administered at a dose of about 4 mg. In another embodiment, one or more compounds represented by the structure of Formula (I) is administered at a dose of about 6 mg. In another embodiment, one or more compounds represented by the structure of Formula (I) is administered at a dose of about 8.4 mg.

In one embodiment, compound (1) is administered at a dose of about 2.4 mg. In another embodiment, compound (1) is administered at a dose of about 4 mg. In another embodiment, compound (1) is administered at a dose of about 6 mg. In one embodiment, compound (2) is administered at a dose of about 2.4 mg. In another embodiment, compound (2) is administered at a dose of about 4 mg. In another embodiment, compound (2) is administered at a dose of about 6 mg.

In one embodiment, compound (22) is administered at a dose of about 0.3 mg. In another embodiment, compound (22) is administered at a dose of about 0.6 mg. In another embodiment, compound (22) is administered at a dose of about 0.9 mg. In another embodiment, compound (22) is administered at a dose of about 1.2 mg. In another embodiment, compound (22) is administered at a dose of about 2 mg. In another embodiment, compound (22) is administered at a dose of about 4 mg.

In one embodiment, the first dose comprises 6 mg of Compound (1) or another compound of Formula (III), and the second dose comprises a lower dosage of Compound (1) or another compound of Formula (III), which in one embodiment, comprises 4 mg of Compound (1) or another compound of Formula (III), and, in another embodiment, comprises 2.4 mg of Compound (1) or another compound of Formula (III). In another embodiment, the third does of Compound (1) or another compound of Formula (III) comprises a lower dosage of Compound (1) or another compound of Formula (III), which in one embodiment, comprises 2.4 mg of Compound (1) or another compound of Formula (III). In one embodiment, the dose of Compound (1) or related compounds is lowered if the subject experiences Grade 4 neutropenia lasting ≥7 days, Grade 3 or 4 febrile neutropenia lasting >24 hours, or Grade 4 thrombocytopenia or ≥Grade 3 thrombocytopenia with significant bleeding.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (i.e., CAPTISOL®), cosolvent solubilization (i.e., propylene glycol) or micellar solubilization (i.e., Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid find use in the preparation of injectables.

A sterile injectable oil-in-water microemulsion can, for example, be prepared by 1) dissolving at least one compound of Formula (I) or Formula (IV) in an oily phase, such as, for example, a mixture of soybean oil and lecithin; 2) combining the Formula (I) or Formula (IV) containing oil phase with a water and glycerol mixture; and 3) processing the combination to form a microemulsion.

A sterile aqueous or oleaginous suspension can be prepared in accordance with methods already known in the art. For example, a sterile aqueous solution or suspension can be prepared with a non-toxic parenterally-acceptable diluent or solvent, such as, for example, 1,3-butane diol; and a sterile oleaginous suspension can be prepared with a sterile non-toxic acceptable solvent or suspending medium, such as, for example, sterile fixed oils, e.g., synthetic mono- or diglycerides; and fatty acids, such as, for example, oleic acid.

Pharmaceutically acceptable carriers, adjuvants, and vehicles that may be used in the pharmaceutical compositions of this invention include, but are not limited to, ion exchangers, alumina, aluminum stearate, lecithin, self-emulsifying drug delivery systems (SEDDS) such as d-alpha-tocopherol polyethyleneglycol 1000 succinate, surfactants used in pharmaceutical dosage forms such as Tweens, polyethoxylated castor oil such as CREMOPHOR® surfactant (BASF), or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-cyclodextrins, or other solubilized derivatives may also be advantageously used to enhance delivery of compounds of the formulae described herein.

The pharmaceutically active compounds of this invention can be processed in accordance with conventional methods of pharmacy to produce medicinal agents for administration to subjects, including humans and other mammals. The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such pharmaceutical compositions may also comprise adjuvants, such as wetting, sweetening, flavoring, and perfuming agents.

The amounts of compounds that are administered and the dosage regimen for treating a disease condition with the first and second compositions of this invention depends on a variety of factors, including the age, weight, gender, the medical condition of the subject, the type of disease, the severity of the disease, the route and frequency of administration, and the particular compound employed. Thus, the dosage regimen may vary widely, but can be determined routinely using standard methods. A daily dose of about 0.001 to 100 mg/kg body weight, preferably between about 0.005 and about 50 mg/kg body weight and most preferably between about 0.01 to 10 mg/kg body weight, may be appropriate.

For therapeutic purposes, the active compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Pharmaceutical compositions of this invention comprise at least one compound of Formula (I) or Formula (IV), or a salt thereof, and optionally an additional agent selected from any pharmaceutically acceptable carrier, adjuvant, and vehicle. Alternate pharmaceutical compositions of this invention comprise a compound of Formula (I) or Formula (IV) described herein, or a prodrug thereof, and a pharmaceutically acceptable carrier, adjuvant, or vehicle.

The compound in accordance with Formula (I) or Formula (IV) can be administered by any means suitable for the condition to be treated, which can depend on the need for site-specific treatment or quantity of Formula (I) or Formula (IV) compound to be delivered. The compounds and pharmaceutical compositions of the present invention may, for example, be administered orally, mucosally, or parentally including intravascularly, intraperitoneally, subcutaneously, intramuscularly, and intrasternally. In one embodiment, the compounds and pharmaceutical compositions of the present invention are administered intravenously. In one embodiment, the compounds and pharmaceutical compositions of the present invention are administered PO.

### Notch-Activating Genetic Alterations

In one embodiment, a cancer as described herein comprises a Notch activating alteration. In another embodiment, a cancer as described herein comprises a Notch activating genetic alteration. In another embodiment, a cancer as described herein comprises a Notch activating mutation. In another embodiment, a cancer as described herein comprises a Notch activating genetic mutation. In another embodiment, a cancer as described herein comprises a Notch mutation. In another embodiment, a cancer as described herein comprises a Notch altering mutation.

In one embodiment, a Notch-activating genetic alteration comprises a mutation in a gene that activates the Notch signaling pathway.

In one embodiment, a Notch-activating genetic alteration comprises a sequence variant of one or more Notch-related genes. In another embodiment, a Notch-activating genetic alteration comprises a mutation in one or more Notch-related genes.

In one embodiment, the mutation in one or more Notch-related genes induces a gain of function (GOF) in Notch activity. In one embodiment, a subject whose cancer cells comprise one or more mutations leading to Notch GOF are administered monotherapy with a compound of Formula (I) as described herein. In another embodiment, a subject whose cancer cells comprise one or more mutations leading to Notch GOF are administered a combination therapy comprising a compound of Formula (I) as described herein and another anti-cancer compound.

In another embodiment, the mutation in one or more Notch-related genes induces a loss of function (LOF) in Notch activity. In one embodiment, a subject whose cancer cells comprise one or more mutations leading to Notch LOF are administered a combination therapy comprising a compound of Formula (I) as described herein and another anti-cancer therapy. In one embodiment, the anti-cancer therapy comprises a chemotherapy.

In another embodiment, it is not known if the mutation is a GOF or LOF Notch mutation. In one embodiment, the mutation comprises a variant of unknown significance (VUS).

In one embodiment, the mutation in one or more Notch-related genes comprises a negative regulatory region (NRR) mutation. In another embodiment, the mutation in one or more Notch-related genes comprises a proline, glutamic acid, serine and threonine rich domain (PEST) mutation. In another embodiment, the mutation in one or more Notch-related genes comprises NRR and PEST mutations.

In one embodiment, the Notch-activating mutation functionally inactivates the PEST domain of the Notch gene. In another embodiment, the Notch-activating mutation functionally inactivates the negative regulatory region (NRR) of the Notch gene.

In one embodiment, the Notch-activating mutation comprises a sequence variant in the NRR domain of a Notch gene. In another embodiment, the Notch-activating mutation comprises a sequence variant in the PEST domain of a Notch gene. In another embodiment, the Notch-activating mutation comprises a sequence variant in both the NRR domain and the PEST domain of one or more Notch genes. In another embodiment, the Notch-activating mutation comprises a gene rearrangement in the ectodomain of a Notch gene. In another embodiment, the gene rearrangement removes most of the ectodomain.

In another embodiment, the gene rearrangement functionally inactivates most of the NRR. In one embodiment, the gene rearrangement removes some of the NRR. In another embodiment, the gene rearrangement removes most of the NRR.

In one embodiment, the Notch-activating mutation is a gain-of-function (GOF) mutation in one or more Notch genes. In one embodiment, such GOF mutations may be associated with the Notch extracellular negative regulatory region (NRR), the Notch intracellular C-terminal PEST degron domain, or both. In one embodiment, the NRR maintains the receptor in the off state in the absence of ligand. In one embodiment, the C-terminal PEST degron domain promotes the rapid turnover of activated Notch receptors.

In one embodiment, a GOF NRR mutation comprises one or more point mutations, one or more in-frame insertions or deletions (indels), one or more gene rearrangements, or a combination thereof. In one embodiment, the mutation perturbs the structure of the NRR. In another embodiment, the mutation removes the coding sequence of the NRR. In one embodiment, the NRR mutation promotes ligand-independent Notch cleavage by ADAMs and/or gamma-secretase, and in one embodiment, generates high levels of NICD. In one embodiment the NRR mutation is in Notch1. In another embodiment, the NRR mutation is in Notch3.

In another embodiment, the GOF mutation may be associated with PEST domain mutations, which, in one embodiment, comprise nonsense mutations, out-of-frame indels, large deletions that remove the PEST domain and sustain the activity of Notch1 Intracellular Domain (NICD1), or a combination thereof.

In one embodiment, the presence of PEST mutations in cis with NRR mutations synergistically increases Notch activation. In one embodiment, the NRR and PEST domain mutations are in a single Notch allele. In another embodiment, the NRR and PEST domain mutations are in different Notch alleles.

In another embodiment, Notch GOF mutations are associated with one or more truncated forms of any one of the four Notch genes. In one embodiment, such truncations comprise rearrangements which, in one embodiment, remove the sequences encoding the ectodomain of the receptor. In one embodiment, these rearrangements produce Notch genes that drive the transcription of aberrant 5′-deleted transcripts encoding constitutively active polypeptides that lack the EGF-like ligand binding domain and/or NRR regions.

In one embodiment the Notch-activating mutation is an NRR mutation described in Weng A P, et al., Science. 2004; 306(5694):269-271 or Stoeck A, et al. Cancer Discov. 2014; 4(10):1154-1167, each of which is herein incorporated by reference in its entirety.

In one embodiment, a mutation in one or more Notch-related genes comprises a mutation in a Notch gene hotspot. In one embodiment, a Notch gene hotspot comprises an NRR domain, a PEST domain, or a combination thereof. In one embodiment, a mutation in one or more Notch-related genes comprises a mutation in an NRR. In another embodiment, a mutation in one or more Notch-related genes comprises a mutation in a PEST domain. In another embodiment, a mutation in one or more Notch-related genes comprises a mutation in an NRR and a PEST domain. In one embodiment, these mutations are GOF mutations.

In another embodiment, the mutation in one or more Notch-related genes comprises a gene rearrangement that removes most of the Notch ectodomain, including the NRR. In one embodiment, these mutations are GOF mutations.

In another embodiment, the Notch-activating genetic alteration comprises a missense mutation. In another embodiment, the Notch-activating genetic alteration comprises a nonsense mutation. In another embodiment, the Notch-activating genetic alteration comprises an insertion. In another embodiment, the Notch-activating genetic alteration comprises a deletion. In another embodiment, the Notch-activating genetic alteration comprises a duplication. In another embodiment, the Notch-activating genetic alteration comprises a frameshift mutation. In another embodiment, the Notch-activating genetic alteration comprises a repeat expansion. In another embodiment, Notch-activating genetic alteration comprises a gene fusion.

In one embodiment, the Notch-related gene comprises a Notch1-related gene. In another embodiment, the Notch-related gene comprises a Notch2-related gene. In another embodiment, the Notch-related gene comprises a Notch3-related gene. In another embodiment, the Notch-related gene comprises a Notch4-related gene.

In another embodiment, the Notch-related gene comprises Notch1. In another embodiment, the Notch-related gene comprises Notch2. In another embodiment, the Notch-related gene comprises Notch3. In another embodiment, the Notch-related gene comprises Notch4.

In one embodiment, the Notch-activating mutation comprises a Notch 1 mutation, a Notch 2 mutation, a Notch 3 mutation, a Notch 4 mutation, or a combination thereof.

In one embodiment, the tumor, cancer, carcinoma, or hyperproliferative disorder comprises a Notch GOF mutation and/or a Notch-activating genetic alteration. In another embodiment, the cancer, carcinoma, or hyperproliferative disorder lacks a Notch GOF mutation and/or a Notch-activating genetic alteration.

### Definitions

Unless specifically stated otherwise herein, references made in the singular may also include the plural. For example, “a” and “an” may refer to either one, or one or more.

The definitions set forth herein take precedence over definitions set forth in any patent, patent application, and/or patent application publication incorporated herein by reference.

Listed below are definitions of various terms used to describe the present invention. These definitions apply to the terms as they are used throughout the specification (unless they are otherwise limited in specific instances) either individually or as part of a larger group.

As used herein, the term “administering” refers to bringing in contact with a compound of the present invention. In one embodiment, the compositions are applied locally. In another embodiment, the compositions are applied systemically. Administration can be accomplished to cells or tissue cultures, or to living organisms, for example humans.

As used herein, the terms “administering,” “administer,” or “administration” refer to delivery of one or more compounds or compositions to a subject parenterally, enterally, or topically. Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration. In particular embodiments, an agent or composition is administered parenterally, optionally by intravenous administration or oral administration to a subject.

In one embodiment, a composition of the present invention comprises a pharmaceutically acceptable composition. In one embodiment, the phrase “pharmaceutically acceptable” is employed herein to refer to those compounds, materials, compositions, combinations, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In one embodiment, a composition or combination of the present invention is administered in a therapeutically effective amount. In one embodiment, a “therapeutically effective amount” is intended to include an amount of a compound of the present invention alone or an amount of the combination of compounds claimed or an amount of a compound of the present invention in combination with other active ingredients effective to act as an inhibitor to a NOTCH receptor, effective to inhibit gamma secretase, or effective to treat or prevent hyperproliferative disorders such as cancer. In one embodiment, a “therapeutically effective amount” of a composition of the invention is that amount of composition which is sufficient to provide a beneficial effect to the subject to which the composition is administered.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

In one embodiment, “treating” refers to, in one embodiment, therapeutic treatment and, in another embodiment, prophylactic or preventative measures. In one embodiment, the goal of treating is to prevent or lessen the targeted pathologic condition or disorder as described hereinabove. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging subject survival, or a combination thereof.

In one embodiment, the term “decreasing the size of the tumor” as used herein is assessed using the “Response Evaluation Criteria In Solid Tumors” (RECIST). In one embodiment, RECIST measures reduction in tumor size by measuring the longest dimension of a target lesion. In one embodiment, the target lesion is selected on the basis of its size (lesion with the longest diameter) and its suitability for accurate repeated measurements (either by imaging techniques or clinically). In one embodiment, all other lesions (or sites of disease) are identified as non-target lesions and are also recorded at baseline. Measurements of these lesions are not required, but the presence or absence of each is noted throughout follow-up.

In one embodiment, the term “decreasing the volume of the tumor” as used herein is assessed using the radiological tumor response evaluation criteria. In one embodiment, the maximum diameter (width) of the tumor is measured in two dimensions in the translation plane and its largest perpendicular diameter on the same image (thickness), according to the World Health Organization (WHO).

According to any of the methods of the present invention and in one embodiment, a subject as described herein is human. In another embodiment, the subject is a mammal. In another embodiment, the subject is a primate, which in one embodiment, is a non-human primate. In another embodiment, the subject is murine, which in one embodiment is a mouse, and, in another embodiment is a rat. In another embodiment, the subject is canine, feline, bovine, equine, caprine, ovine, porcine, simian, ursine, vulpine, or lupine. In one embodiment, the subject is a chicken or fish.

In one embodiment, the compositions as described herein comprise the components of the composition (i.e., one or more compounds of Formula (I)) as described herein. In another embodiment, the compositions as described herein consist of the components of the composition (i.e., one or more compounds of Formula (I)) as described herein). In another embodiment, the compositions as described herein consist essentially of the components of the composition (i.e., one or more compounds of Formula (I)) as described herein.

It is to be understood that the compositions, combinations and methods of the present invention comprising the elements or steps as described herein may, in another embodiment, consist of those elements or steps, or in another embodiment, consist essentially of those elements or steps. In some embodiments, the term “comprise” refers to the inclusion of the indicated active agents, such as the gamma secretase inhibitor, as well as inclusion of other active agents, and pharmaceutically or physiologically acceptable carriers, excipients, emollients, stabilizers, etc., as are known in the pharmaceutical industry. In some embodiments, the term “consisting essentially of” refers to a composition, whose only active ingredients are the indicated active ingredients. However, other compounds may be included which are for stabilizing, preserving, etc. the formulation, but are not involved directly in the therapeutic effect of the indicated active ingredients. In some embodiments, the term “consisting essentially of” may refer to components which facilitate the release of the active ingredient. In some embodiments, the term “consisting” refers to a composition, which contains the active ingredients and a pharmaceutically acceptable carrier or excipient.

### Timing and Site of Administration

In some embodiments, one or more compositions of the present invention are administered at least once during a treatment cycle. In some embodiments, the compositions of the present invention are administered to the subject on the same days. In some embodiments, the compositions of the present invention are administered to the subject on the different days. In some embodiments, one or more compositions of the present invention are administered to the subject on the same days and on different days according to treatment schedules.

In particular embodiments, one or more compositions of the present invention are administered to the subject over one or more treatment cycles

In some embodiments, the treatment cycle comprises administration of the composition on 2 consecutive days followed by 5 consecutive days with no administration (2 days on/5 days off). In some embodiments, the treatment cycle comprises administration of the composition on 3 consecutive days followed by 4 consecutive days with no administration (3 days on/4 days off). In some embodiments, the treatment cycle comprises administration of the composition on 4 consecutive days followed by 3 consecutive days with no administration (4 days on/3 days off). In some embodiments, the treatment cycle comprises administration of the composition on 5 consecutive days followed by 2 consecutive days with no administration (5 days on/2 days off).

In other embodiments, a treatment cycle can be at least two, at least three, at least four, at least five, at least six, at least seven, at least 14, at least 21, at least 28, at least 48, or at least 96 days or more. In one embodiment, a treatment cycle is 28 days. In certain embodiments, the compositions are administered over the same treatment cycle or concurrently over different treatment cycles assigned for each composition. In various embodiments, the treatment cycle is determined by a health care professional based on conditions and needs of the subject.

In some embodiments, a composition is administered on at least one day, at least two days, at least three days, at least four days, at least five days, at least six days, at least seven days, at least eight days, at least nine days, at least ten days, at least eleven days, at least twelve days, at least 13 days, at least 14 days, at least 21 days, or all 28 days of a 28 day treatment cycle. In particular embodiments, a composition is administered to a subject once a day. In other particular embodiments, a composition is administered twice a day.

In some embodiments, in the methods of the present invention, a composition as described herein comprising a compound of Formula (I) is administered once per week. In some embodiments, in the methods of the present invention, a composition as described herein comprising one or more compounds of Formula (I) is administered once every two weeks. In one embodiment, compound (22) or compound (1) or compound (2) is administered once per week. In another embodiment, compound (22) or compound (1) or compound (2) is administered once every two weeks.

In some embodiments, in the methods of the present invention, a composition as described herein comprising one or more compounds of Formula (I) is administered once per day. In some embodiments, in the methods of the present invention, a composition as described herein comprising one or more compounds of Formula (I) is administered intermittently. In some embodiments, the intermittent administration comprises two days on/five days off. In other embodiments, the intermittent administration comprises three days on/four days. In one embodiment, compound (22) is administered once per day. In another embodiment, compound (22) is administered intermittently. In another embodiment, compound (22) is administered two days on/five days off. In another embodiment, compound (22) is administered three days on/four days.

In one embodiment, one or more of the compositions described herein are administered in one to four doses per day. In one embodiment, one or more of the compositions described herein is administered once per day. In another embodiment, one or more of the compositions described herein is administered twice per day. In another embodiment, one or more of the compositions described herein is administered three times per day. In another embodiment, one or more of the compositions described herein is administered four times per day. In another embodiment, one or more of the compositions described herein is administered once every two days, once every three days, twice a week, once a week, once every 2 weeks, once every 3 weeks.

In some embodiments, in the methods of the present invention, a composition as described herein comprising one or more compounds of Formula (I) are orally administered to the subject. In some embodiments, in the methods of the present invention, a composition as described herein comprising one or more compounds of Formula (I) are intravenously administered to the subject.

In one embodiment, one or more of the compositions described herein is administered for 7 days to 28 days. In another embodiment, one or more of the compositions described herein is administered for 7 days to 8 weeks. In another embodiment, one or more of the compositions described herein is administered for 7 days to 50 days. In another embodiment, one or more of the compositions described herein is administered for 7 days to six months. In another embodiment, one or more of the compositions described herein is administered for 7 days to one and half years. In another embodiment, one or more of the compositions described herein is administered for 14 days to 12 months. In another embodiment, one or more of the compositions described herein is administered for 14 days to 3 years. In another embodiment, one or more of the compositions described herein is administered for several years. In another embodiment, one or more of the compositions described herein is administered for one month to six months.

In one embodiment, one or more of the compositions described herein is administered for 7 days. In another embodiment, one or more of the compositions described herein is administered for 14 days. In another embodiment, one or more of the compositions described herein is administered for 21 days. In another embodiment, one or more of the compositions described herein is administered for 28 days. In another embodiment, one or more of the compositions described herein is administered for 50 days. In another embodiment, one or more of the compositions described herein is administered for 56 days. In another embodiment, one or more of the compositions described herein is administered for 84 days. In another embodiment, one or more of the compositions described herein is administered for 90 days. In another embodiment, one or more of the compositions described herein is administered for 120 days.

The number of times a composition is administered to a subject in need thereof depends on the discretion of a medical professional, the disorder, the severity of the disorder, and the subject's response to the formulation. In some embodiments, the compositions disclosed herein are administered once to a subject in need thereof with a mild acute condition. In some embodiments, the compositions disclosed herein are administered more than once to a subject in need thereof with a moderate or severe acute condition. In the case wherein the subject's condition does not improve, upon the doctor's discretion the composition may be administered chronically, that is, for an extended period of time, including throughout the duration of the subject's life in order to ameliorate or otherwise control or limit the symptoms of the subject's disease or condition.

In the case wherein the subject's status does improve, upon the doctor's discretion the composition may administered continuously; or the dose of drug being administered may be temporarily reduced or temporarily suspended for a certain length of time (i.e., a “drug holiday”). The length of the drug holiday varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

In some embodiments, in the methods of the present invention, the first composition comprising one or more compounds of Formula (I) and the second composition are administered together. In some embodiments, in the methods of the present invention, the first composition comprising one or more compounds of Formula (I) and the second composition are administered at separate sites or at separate times. In some embodiments, in the methods of the present invention, the first composition comprising one or more compounds of Formula (I) and the second composition are administered at separate sites. In some embodiments, in the methods of the present invention, the first composition comprising one or more compounds of Formula (I) and the second composition are administered at separate times.

In one embodiment, in the methods of the present invention, the administration of a second composition occurs prior to the administration of the first composition comprising one or more compounds of Formula (I) or Formula (IV). In another embodiment, in the methods of the present invention, the administration of a second composition occurs following the administration of the first composition comprising one or more compounds of Formula (I) or Formula (IV). In one embodiment, concurrent administration comprises administering a single combination comprising the second composition and the first composition comprising one or more compounds of Formula (I) or Formula (IV). In another embodiment, concurrent administration comprises administering separate compositions.

### Kits

The present invention further comprises the compositions of the present invention and, optionally, one or more additional agents in kit form, e.g., where they are packaged together or placed in separate packages to be sold together as a kit, or where they are packaged to be formulated together.

In certain embodiments, the kit comprises a therapeutic or prophylactic composition containing an effective amount of the compound of Formula (I) or Formula (IV) or Compound (22), as described herein, which in one embodiment, comprises 2 mg or 4 mg of the compound of Formula (IV), and optionally a second composition comprising one or more therapeutic agents. In certain embodiments, the kit comprises a sterile container which contains therapeutic or prophylactic agents; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some embodiments, the one or more therapeutic agents comprises an HDAC inhibitor, a Bcl2 inhibitor, a CDK 4/6 inhibitor, a tyrosine kinase inhibitor, a FGFR inhibitor, an inhibitor of PRMT5, an inhibitor of Bromodomain and BET, a retinoic acid, or a combination thereof. In other embodiments, the one or more therapeutic agents comprises panobinostat, romidepsin, vorinostat, belinostat, venetoclax, lisaftoclax, (APG-2575), palbociclib, ribociclib, abemaciclib, pazopanib, sorafenib, lenvatinib, regorafenib, rivoceranib (apatinib), erdafitinib, pemigatinib, or infigratinib.

If desired, the composition(s) are provided together with instructions for administering the composition(s) to a subject having or at risk of developing a hyperproliferative disorder. The instructions will generally include information about the use of the composition for the treatment or prevention of a hyperproliferative disorder. In other embodiments, the instructions include at least one of the following: description of the therapeutic agent; dosage schedule and administration for treatment or prevention of a hyperproliferative disorder or symptoms thereof, precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

In another embodiment, the present invention further provides a kit for identifying a candidate subject for treatment with a first composition comprising one or more compounds represented by the structure of Formula (III), (IV), (1), (2) or (22), as described herein, and, optionally, a second composition comprising one or more therapeutic agents, and further comprising an evaluator of Notch gene function. In another embodiment, other methods of evaluating the quantity of downstream Notch protein RNA may be utilized, as are well known in the art. In another embodiment, the evaluator comprises a DNA sequencing method, as are known in the art. In one embodiment, instructions for use are included in the kit.

In another embodiment, the present invention further provides a kit for treating a Desmoid tumor in a subject comprising a) an evaluator of Notch gene function b) a first composition comprising one or more compounds represented by the structure of Formula (III), (IV), (1), (2) or (22), as described herein. In one embodiment, the evaluator comprises RNA-seq or another RNA sequencing tool to reveal the presence and quantity of RNA in a biological sample at a given moment. In another embodiment, other methods of evaluating the quantity of downstream Notch protein RNA may be utilized, as are well known in the art. In another embodiment, the evaluator comprises a DNA sequencing method, as are known in the art. In one embodiment, instructions for use are included in the kit.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

It should be understood that the disclosure presented herein is not limited to the particular methodologies, protocols and reagents, and examples described herein. The terminology and examples used herein is for the purpose of describing particular embodiments only, for the intent and purpose of providing guidance to the skilled artisan, and is not intended to limit the scope of the disclosure presented herein.

### Examples

### Methods

The pivotal Phase 2/3 study is a randomized multi-center trial initiated in March 2021. Part A of the study is evaluating the efficacy, safety, tolerability, and tumor volume by MRI after 16 weeks of Compound (22) in adult and adolescent patients with desmoid tumors. It enrolled 42 patients and is evaluating 3 doses of Compound (22). Patients who participated in Part A will be eligible to enroll into an open-label extension study at the selected dose, and long-term efficacy and safety will be monitored. Part B of the study will be double-blind, placebo-controlled, and will start immediately after dose selection from part A enrolling up to 156 patients with progressive disease, randomized between Compound (22) or placebo. The study's primary endpoint will be progression-free survival (PFS) with secondary endpoints including objective response rate (ORR), duration of response (DOR), and patient-reported Quality of Life (QOL) measures.

### Phase 2/3 Clinical Trial Evaluating Compound (22) in Desmoid Tumors Interim Results as of Jul. 14, 2022

### Part A Efficacy

Patient enrollment in Part A of the clinical trial was completed in February 2022. Patients were dosed in Compound (22) monotherapy cohorts of 1.2 mg (daily), 2 mg (2 days on, 5 days off), or 4 mg (2 days on, 5 days off).

The activity of Compound (22) is being evaluated by change in tumor volume (central MRI readings) and response (per RECIST 1.1) determined by blinded independent central review. At data cut, 28 patients were evaluable for tumor volume and 29 were evaluable for RECIST with a scan at base line and at least one additional scan at week 16.

12 subjects had follow up MRI scans at week 28 and one patient had a scan at week 40.

One patient had a partial response (PR) per RECIST at week 16, confirmed at week 28.

Three additional unconfirmed PRs were observed, two at week 28 and one at week 40.

Continuous tumor reduction was observed over time in all patients that underwent 2 or more MRI scans both per RECIST and tumor volume.

### Part A Efficacy of Selected Dose of 1.2 mg Daily:

At week 16, there were 9 evaluable patients for RECIST in the selected dose of 1.2 mg daily with one PR observed, confirmed at week 28. The remaining 8 patients had stable disease, of which 6 patients had a tumor reduction.

At week 28, there were three patients evaluable for RECIST in the selected dose of 1.2 mg daily with one confirmed and one unconfirmed PR and one stable disease with all patients showing tumor reduction and deepening of tumor shrinkage since previous scan.

At week 16, there were 9 evaluable patients for volume change with 7 patients experiencing tumor volume reduction. At week 28, there were three evaluable patients for volume change in the selected dose of 1.2 mg daily with all three patients experiencing continuous tumor shrinkage.

### Part A Safety:

Compound (22) was generally well tolerated at all doses

Most adverse events were grade 1 or 2 and included mainly diarrhea

No grade 4 or 5 events were observed

Amenorrhea was observed in four patients with one patient at the selected dose

Preliminary evidence of AMH level improvement was observed after discontinuation of Compound (22)

Compound (22) monotherapy demonstrated anti-tumor activity in patients with desmoid tumors within a 16-week period and was well tolerated across all doses, which could allow for long term treatment of patients. Compound (22) has the potential to significantly improve the lives of patients suffering from desmoid tumors who currently have no approved therapy.

Efficacy was demonstrated across all cohorts of Part A of the study, with early responses that deepened over time.

What is claimed is:

1. A method of treating a Desmoid tumor or inhibiting the growth of a Desmoid tumor in a subject comprising the step of administering to said subject a composition comprising one or more compounds represented by the structure of Formula (I): 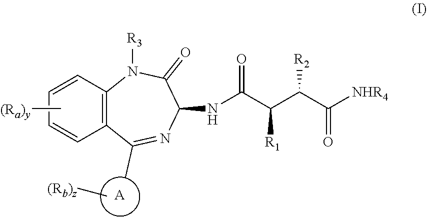
and/or at least one salt thereof, wherein:
R₁ is —CH₂CF₃ or —CH₂CH₂CF₃;
R₂ is —CH₂CF₃, —CH₂CH₂CF₃, or —CH₂CH₂CH₂CF₃;
R₃ is H, —CH₃ or Rx;
R₄ is H or R_{y};
Rₓ
is: —CH₂OC(O)CH(CH₃)NH₂, —CH₂OC(O)CH(NH₂)CH(CH₃)₂, —CH₂OC(O)CH((CH(CH₃)₂)NHC(O)CH(NH₂)CH(CH₃)₂, 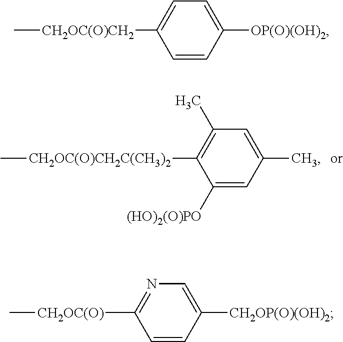
R_{y} is: —SCH₂CH(NH₂)C(O)OH, —SCH₂CH(NH₂)C(O)OH₃,
or —SCH₂CH(NH₂)C(O)OC(CH₃)₃;
Ring A is phenyl or pyridinyl;
each Rₐ is independently F, Cl, —CN, —OCH₃, C₁₋₃ alkyl, —CH₂OH, —CF₃, cyclopropyl, —OCH₃, —O(cyclopropyl) and/or —NHCH₂CH₂OCH₃;
each R_{b} is independently F, Cl, —CH₃, —CH₂OH, —CF₃, cyclopropyl, and/or —OCH₃;
y is zero, 1 or 2; and
z is 1 or 2.

2. The method of claim 1, wherein said composition is administered at a dose of 1.2 mg, 2 mg, or 4 mg.

3. The method of claim 2, wherein said 1.2 mg dose is administered daily.

4. The method of claim 2, wherein said 2 mg or 4 mg dose is administered intermittently.

5. The method of claim 4, wherein said intermittent administration comprises 2 days on, 5 days off.

6. The method of claim 1, wherein Ring A is phenyl; and R₃ is H. In another embodiment, R₂ is —CH₂CH₂CF₃; and Ring A is phenyl. I

7. The method of claim 1, wherein R₂ is —CH₂CH₂CF₃; Ring A is phenyl; Rₐ is C₁₋₃ alkyl or —CH₂OH; each R_{b} is independently F and/or Cl; and y is 1.

8. The method of claim 1, wherein the compound of Formula (I) comprises one or more compounds represented by the structure of Formula (IV):

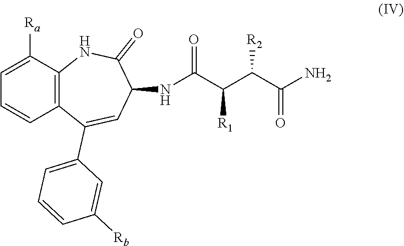

9. The method of claim 1, wherein the compound of Formula (I) comprises one or more compounds represented by the structure of Formula (V): 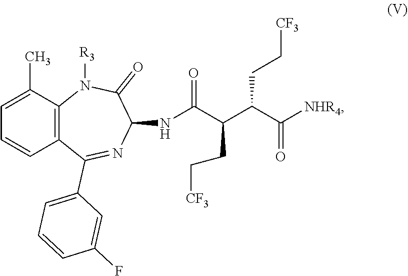
wherein R₃ is H or Rₓ.

10. The method of claim 1, wherein the compound of Formula (I) comprises (2R,3S)—N-((3S)-5-(3-fluorophenyl)-9-methyl-2-oxo-2,3-dihydro-1H-1,4-benzodiazepin-3-yl)-2,3-bis(3,3,3-trifluoropropyl)succinamide (22); (2R,3S)—N-((3S)-5-(3-chlorophenyl)-9-ethyl-2-oxo-2,3-dihydro-1H-1,4-benzodiazepin-3-yl)-2,3-bis(3,3,3-trifluoropropyl)succinamide (23); (2R,3S)—N-((3S)-5-(3-chlorophenyl)-9-isopropyl-2-oxo-2,3-dihydro-1H-1,4-benzodiazepin-3-yl)-2,3-bis(3,3,3-trifluoropropyl)succinamide (24); (2R,3S)—N-(9-chloro-5-(3,4-dimethylphenyl)-2-oxo-2,3-dihydro-1H-1,4-benzodiazepin-3-yl)-3-(4,4,4-trifluorobutyl)-2-(3,3,3-trifluoropropyl)succinamide (25); (2R,3S)—N-(9-chloro-5-(3,5-dimethylphenyl)-2-oxo-2,3-dihydro-1H-1,4-benzodiazepin-3-yl)-3-(4,4,4-trifluorobutyl)-2-(3,3,3-trifluoropropyl)succinamide (26); (2R,3S)—N-((3S)-9-ethyl-5-(3-methylphenyl)-2-oxo-2,3-dihydro-1H-1,4-benzodiazepin-3-yl)-2,3-bis(3,3,3-trifluoropropyl)succinamide (27); (2R,3S)—N-((3S)-5-(3-chlorophenyl)-9-methyl-2-oxo-2,3-dihydro-1H-1,4-benzodiazepin-3-yl)-2,3-bis(3,3,3-trifluoropropyl)succinamide (28); (2R,3S)—N-((3S)-5-(3-chlorophenyl)-9-methyl-2-oxo-2,3-dihydro-1H-1,4-benzodiazepin-3-yl)-3-(4,4,4-trifluorobutyl)-2-(3,3,3-trifluoropropyl)succinamide (29); (2R,3S)—N-((3S)-5-(3-methylphenyl)-2-oxo-9-(trifluoromethyl)-2,3-dihydro-1H-1,4-benzodiazepin-3-yl)-2,3-bis(3,3,3-trifluoropropyl)succinamide (30); (2R,3S)—N-((3S)-9-chloro-5-(3,5-dimethylphenyl)-2-oxo-2,3-dihydro-1H-1,4-benzodiazepin-3-yl)-2,3-bis(3,3,3-trifluoropropyl)succinamide (31); (2R,3S)—N-((3S)-5-(3-methylphenyl)-2-oxo-9-(trifluoromethyl)-2,3-dihydro-1H-1,4-benzodiazepin-3-yl)-3-(4,4,4-trifluorobutyl)-2-(3,3,3-trifluoropropyl)succinamide (32); (2R,3S)—N-((3S)-9-isopropyl-5-(3-methylphenyl)-2-oxo-2,3-dihydro-1H-1,4-benzodiazepin-3-yl)-2,3-bis(3,3,3-trifluoropropyl)succinamide (33); (2R,3S)—N-((3S)-9-(cyclopropyloxy)-5-(3-methylphenyl)-2-oxo-2,3-dihydro-1H-1,4-benzodiazepin-3-yl)-3-(4,4,4-trifluorobutyl)-2-(3,3,3-trifluoropropyl)succinamide (34); (2R,3S)—N-((3S)-9-(cyclopropyloxy)-5-(3-methylphenyl)-2-oxo-2,3-dihydro-1H-1,4-benzodiazepin-3-yl)-2,3-bis(3,3,3-trifluoropropyl)succinamide (35); (2R,3S)—N-((3S)-9-chloro-5-(3-methylphenyl)-2-oxo-2,3-dihydro-1H-1,4-benzodiazepin-3-yl)-3-(4,4,4-trifluorobutyl)-2-(3,3,3-trifluoropropyl) succinamide (36); (2R,3S)—N-((3S)-9-methyl-2-oxo-5-(3-(trifluoromethyl)phenyl)-2,3-dihydro-1H-1,4-benzodiazepin-3-yl)-3-(4,4,4-trifluorobutyl)-2-(3,3,3-trifluoropropyl) succinamide (37); (2R,3S)—N-((3S)-9-methyl-2-oxo-5-(3-(trifluoromethyl) phenyl)-2,3-dihydro-1H-1,4-benzodiazepin-3-yl)-2,3-bis(3,3,3-trifluoropropyl) succinamide (38); (2R,3S)—N-((3S)-9-chloro-5-(2-methylphenyl)-2-oxo-2,3-dihydro-1H-1,4-benzodiazepin-3-yl)-2,3-bis(3,3,3-trifluoropropyl)succinamide (39); (2R,3S)—N-((3S)-5-(4-fluorophenyl)-9-methyl-2-oxo-2,3-dihydro-1H-1,4-benzodiazepin-3-yl)-2,3-bis(3,3,3-trifluoropropyl)succinamide (40); (2R,3S)—N-((3S)-9-chloro-5-(3-cyclopropylphenyl)-2-oxo-2,3-dihydro-1H-1,4-benzodiazepin-3-yl)-2,3-bis(3,3,3-trifluoropropyl)succinamide (41); (2R,3S)—N-((3S)-5-(3-chlorophenyl)-9-methoxy-2-oxo-2,3-dihydro-1H-1,4-benzodiazepin-3-yl)-2,3-bis(3,3,3-trifluoropropyl)succinamide (42); (2R,3S)—N-((3S)-5-(4-chlorophenyl)-9-methoxy-2-oxo-2,3-dihydro-1H-1,4-benzodiazepin-3-yl)-2,3-bis(3,3,3-trifluoropropyl)succinamide (43); (2R,3S)—N-((3S)-9-chloro-5-(3-methylphenyl)-2-oxo-2,3-dihydro-1H-1,4-benzodiazepin-3-yl)-2,3-bis(3,3,3-trifluoropropyl)succinamide (44); (2R,3S)—N-((3S)-5-(3-methylphenyl)-9-methoxy-2-oxo-2,3-dihydro-1H-1,4-benzodiazepin-3-yl)-2,3-bis(3,3,3-trifluoropropyl)succinamide (45); (2R,3S)—N-((3S)-5-(4-(hydroxymethyl)phenyl)-2-oxo-2,3-dihydro-1H-1,4-benzodiazepin-3-yl)-2,3-bis(3,3,3-trifluoropropyl)succinamide (46); (2R,3S)—N-((3S)-5-(2-methylphenyl)-2-oxo-2,3-dihydro-1H-1,4-benzodiazepin-3-yl)-2,3-bis(3,3,3-trifluoropropyl)succinamide (47); (2R,3S)—N-((3S)-5-(3-methylphenyl)-2-oxo-2,3-dihydro-1H-1,4-benzodiazepin-3-yl)-2,3-bis(3,3,3-trifluoropropyl)succinamide (48); (2R,3S)—N-((3S)-9-methoxy-2-oxo-5-(5-(trifluoromethyl)-2-pyridinyl)-2,3-dihydro-1H-1,4-benzodiazepin-3-yl)-2,3-bis(3,3,3-trifluoropropyl)succinamide (49); (2R,3S)—N-((3S)-5-(5-chloro-2-pyridinyl)-9-methoxy-2-oxo-2,3-dihydro-1H-1,4-benzodiazepin-3-yl)-2,3-bis(3,3,3-trifluoropropyl)succinamide (50); (2R,3S)—N-((3S)-5-(4-methoxyphenyl)-2-oxo-2,3-dihydro-1H-1,4-benzodiazepin-3-yl)-2,3-bis(3,3,3-trifluoropropyl)succinamide (51); (2R,3S)—N-((3S)-5-(4-methylphenyl)-2-oxo-2,3-dihydro-1H-1,4-benzodiazepin-3-yl)-2,3-bis(3,3,3-trifluoropropyl)succinamide (52); (2R,3S)—N-((3S)-5-(3-fluorophenyl)-9-(hydroxymethyl)-2-oxo-2,3-dihydro-1H-1,4-benzodiazepin-3-yl)-2,3-bis(3,3,3-trifluoropropyl)succinamide (53); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valinate (54); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-alaninate (55); S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteine (56); tert-butyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl)hexanoyl)amino)-L-cysteinate (57); methyl S-(((2S,3R)-6,6,6-trifluoro-3-(((3S)-5-(3-fluorophenyl)-9-methyl-2-oxo-2,3-dihydro-1H-1,4-benzodiazepin-3-yl)carbamoyl)-2-(3,3,3-trifluoropropyl) hexanoyl)amino)-L-cysteinate (58); ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl (4-(phosphonooxy)phenyl)acetate (59); and ((3S)-3-(((2R,3S)-3-carbamoyl-6,6,6-trifluoro-2-(3,3,3-trifluoropropyl)hexanoyl)amino)-5-(3-fluorophenyl)-9-methyl-2-oxo-2,3-dihydro-1H-1,4-benzodiazepin-1-yl)methyl L-valyl-L-valinate (60); and salts thereof.

11. The method of claim 1, wherein the compound of Formula (I) comprises:

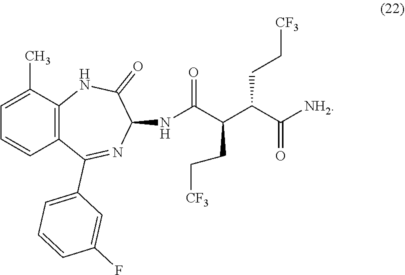 [SMILES: Cc1cccc2c1NC(=O)[C@@H](NC(=O)[C@H](CCC(F)(F)F)[C@H](CCC(F)(F)F)C(N)=O)N=C2c1cccc(F)c1]

12. The method of claim 1, wherein said composition is orally administered to said subject.

13. The method of claim 1, further comprising the step of administering a composition comprising an additional treatment.

14. The method of claim 12, wherein said first composition and said second composition are administered together.

15. The method of claim 12, wherein said first composition and said second composition are administered at separate sites or at separate times.

16. The method of claim 1, wherein said Desmoid tumor comprises a Notch-activating mutation.
